(19) United States
(12) Patent Application Publication
(10) Pub. No.: US 2011/0027331 A1
(43) Pub. Date: Feb. 3, 2011

(54) AN IMPLANTABLE DRUG DEPOT HAVING A REVERSIBLE PHASE TRANSITION MATERIAL FOR TREATMENT OF PAIN AND/OR INFLAMMATION

(75) Inventor: Christopher M. Hobot, Tonka Bay, MN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/511,524

(22) Filed: Jul. 29, 2009

(51) Int. Cl. A61K 9/00; A61K 47/30; A61K 47/38; A61P 29/00

(52) U.S. Cl. 424/422; 514/772.3; 514/781

(57) ABSTRACT

Effective treatments of pain and/or inflammation are provided that utilize a reversible phase transition material of a drug depot. When heat, cold or another suitable form of energy, e.g., ultrasound energy is applied to the reversible phase transition material, the release of an analgesic and/or anti-inflammatory agent from a drug depot is increased.

### BACKGROUND

Pain relief is of prime importance to anyone treating patients undergoing surgery. Proper pain relief imparts significant physiological and psychological benefits to the patient. Not only does effective pain relief mean a smoother more pleasant postoperative course (e.g., mood, sleep, quality of life, etc.) with earlier discharge from medical/surgical/outpatient facilities, but it may also reduce the onset of chronic pain syndromes (e.g., fibromyalgia, myalgia, etc.).

Pain serves a biological function. It often signals the presence of damage or disease within the body and is often accompanied by inflammation (redness, swelling, and/or burning). In the case of postoperative pain, it may be a result of the surgery, or other treatments such as, for example, management of acute pain following burns or non-surgical trauma. The goal for postoperative pain management is to reduce or eliminate pain and discomfort with medication that cause minimum or no side effects.

The site of the surgery has a profound effect upon the degree of postoperative pain a patient may suffer. In general, operations on the thorax and upper abdomen are more painful than operations on the lower abdomen, which in turn are more painful than peripheral operations on the limbs. However, any operation involving a body cavity, large joint surfaces, the spine or deep tissues should be regarded as painful. In particular, operations on the thorax or upper abdomen may produce widespread changes in pulmonary function, an increase in abdominal muscle tone and an associated decrease in diaphragmatic function. The result will be an inability to cough and clear secretions, which may lead to lung collapse and pneumonia. Prolonged pain can reduce physical activity and lead to venous stasis and an increased risk of deep vein thrombosis and consequently pulmonary embolism. In addition, there can be widespread effects on gut and urinary tract motility, which may lead in turn to postoperative ileus, nausea, vomiting and urinary retention. These problems are unpleasant for the patient and may prolong hospital stay. Most patients who experience moderate to severe post-operative pain, post-traumatic pain and burn pain, often require pain control at least in the first 3 days after trauma or surgery.

Unfortunately, currently available pain and/or anti-inflammatory formulations, although effective for treating postoperative pain, require frequent single dose administration every 4 to 12 hours on an as needed basis. Often with the single dose dosing, the patient will experience break through pain and anxious “clock-watching” waiting for the next dose in order to provide persistent pain relief. These single dose formulations are inconvenient and may interfere with the patient's postoperative inpatient and/or outpatient daytime activities and nighttime sleep and recovery.

New analgesic and/or anti-inflammatory compositions and methods are needed to prevent, treat or reduce pain and/or inflammation, particularly post operative pain and/or inflammation. New analgesic and/or anti-inflammatory compositions and methods that reliably reduce, prevent or treat episodes of breakthrough pain, as well as provide long acting analgesic and anti-inflammatory effects over periods of at least one day are needed.

### SUMMARY

Novel compositions and methods are provided for effectively reducing, preventing, or treating unwanted breakthrough pain and/or inflammation. The pain and/or inflammation may be reduced for extended periods of time.

In various embodiments, new drug depot compositions and methods are provided, which can easily allow accurate and precise implantation of a drug depot containing the analgesic and/or anti-inflammatory with minimal physical and psychological trauma to a patient. One advantage of the drug depot composition is that by employing a reversible phase transition material, the patient or practitioner can provide heat, cold or another suitable form of energy, e.g., ultrasound energy, at or near the drug depot so that an increased dose of the analgesic and/or anti-inflammatory agent is released at a target tissue site (e.g., spine, knee, shoulder, hip, abdomen, synovial joint, at or near the spinal column, surgical wound or incision, intraspinally etc.). In this way, for example, breakthrough pain can be effectively reduced, prevented and/or treated.

In one embodiment, an implantable drug depot is provided that is useful for reducing, preventing or treating pain and/or inflammation in a patient in need of such treatment, the implantable drug depot being implantable at a site beneath the skin and comprising an effective amount of an analgesic and/or an anti-inflammatory agent disposed within a reversible phase transition material of the drug depot, wherein the reversible phase transition material is capable of releasing a bolus dose of the analgesic, muscle relaxant and/or the anti-inflammatory agent when heat, cold or other form of energy, e.g., ultrasound energy is applied to the skin of a patient to reduce, prevent or treat pain and/or inflammation.

In another embodiment, a drug depot is provided that is useful for reducing, preventing or treating pain and/or inflammation in a patient in need of such treatment, the drug depot being implantable at a site beneath the skin of the patient and comprising an effective amount of an analgesic and/or an anti-inflammatory agent disposed within a reversible phase transition polymer and a biodegradable polymer of the drug depot, wherein the reversible phase transition material is capable of causing the drug depot to release a bolus dose of the analgesic, muscle relaxant and/or the anti-inflammatory agent when heat, cold or other form on energy is applied at or near the drug depot and the biodegradable polymer is capable of releasing the analgesic, muscle relaxant and/or the anti-inflammatory agent over at least one day to reduce, prevent or treat pain and/or inflammation.

In yet another embodiment, a method is provided for treating or preventing pain and/or inflammation in a patient in need of such treatment, the method comprising implanting at a target tissue site beneath the skin of patient a biodegradable drug depot comprising an effective amount of an analgesic and/or an anti-inflammatory agent disposed within a reversible phase transition material of the drug depot, wherein the reversible phase transition material is capable of releasing a bolus dose of the analgesic, muscle relaxant and/or the anti-inflammatory agent when heat, cold or another suitable form of energy, e.g., ultrasound energy, light, mechanical energy (such as agitation), electrical, chemical, or magnetic energy is applied to or near the drug depot; and applying heat, cold or another suitable form of energy, e.g., ultrasound energy, to or near the target tissue site where the drug depot is implanted to release the bolus dose of the analgesic, muscle relaxant and/or the anti-inflammatory agent to prevent or treat pain and/or inflammation.

The compositions and methods provided may be used to reduce, prevent, or treat inflammation and/or pain, including but not limited to inflammation and/or pain that follows surgery, chronic inflammatory diseases, chronic pelvic pain syndromes (e.g., interstitial cystitis, chronic non-bacterial prostatitis, vulvodynia, endometriosis, irritable bowel disease and other conditions that result in chronic pain in the pelvic region), bursitis, osteoarthritis, osteolysis, tendonitis, sciatica, herniated discs, stenosis, myopathy, spondilothesis, lower back pain, facet pain, carpal tunnel syndrome, tarsal tunnel syndrome, failed back pain or the like.

The pharmaceutical composition may for example, be part of a drug depot. The drug depot may: (i) consist of the analgesic and/or anti-inflammatory agent and the reversible phase transition material and/or the biodegradable polymer(s); or (ii) consist essentially of the analgesic and/or anti-inflammatory agent and the reversible phase transition material and/or the biodegradable polymer(s); or (iii) comprise the analgesic and/or anti-inflammatory agent and the reversible phase transition material and/or the biodegradable polymer(s) and one or more other active ingredients, surfactants, excipients or other ingredients or combinations thereof. When there are other active ingredients, surfactants, excipients or other ingredients or combinations thereof in the formulation, in some embodiments these other compounds or combinations thereof comprise less than 20 wt. %, less than 19 wt. %, less than 18 wt. %, less than 17 wt. %, less than 16 wt. %, less than 15 wt. %, less than 14 wt. %, less than 13 wt. %, less than 12 wt. %, less than 11 wt. %, less than 10 wt. %, less than 9 wt. %, less than 8 wt. %, less than 7 wt. %, less than 6 wt. %, less than 5 wt. %, less than 4 wt. %, less than 3 wt. %, less than 2 wt. %, less than 1 wt. % or less than 0.5 wt. %.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 is a magnified side sectional view of an embodiment of the implantable drug depot having a layer of the reversible phase transition material holding the analgesic and/or anti-inflammatory agent within the drug depot.

FIG. 2 is a magnified side sectional view of an embodiment of the implantable drug depot having a layer of the reversible phase transition material that is changing to a reduced viscosity or increased permeability state causing release of the analgesic and/or anti-inflammatory agent from the drug depot as heat, cold or another suitable form of energy, e.g., ultrasound energy, is applied to it.

FIG. 3 is a perspective view of one embodiment illustrating a cold or hot pack being applied to the skin near the area that the drug depot was implanted. The application of cold or heat causes the reversible phase transition material to reversibly change phases (e.g., solid to liquid, solid to semi-solid, semi-solid to liquid, water-insoluble to water soluble, glassy to rubbery, crystalline or semi-crystalline to liquid, etc.) to release a bolus dose of the analgesic and/or anti-inflammatory agent.

FIG. 4 illustrates a number of common locations within a patient that may be sites at which pain and/or inflammation can occur and locations at which a drug depot containing an analgesic, muscle relaxant and/or the anti-inflammatory agent can locally be administered thereto.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding the numerical ranges and parameters set forth herein, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

The headings below are not meant to limit the disclosure in any way; embodiments under any one heading may be used in conjunction with embodiments under any other heading.

It is noted that, as used in this specification and the appended claims, the singular forms “a,” “an,” and “the,” include plural referents unless expressly and unequivocally limited to one referent. Thus, for example, reference to “a drug depot” includes one, two, three or more drug depots.

The abbreviation “DLG” refers to poly(DL-lactide-co-glycolide).

The abbreviation “DL” refers to poly(DL-lactide).

The abbreviation “LG” refers to poly(L-lactide-co-glycolide).

The abbreviation “CL” refers to polycaprolactone.

The abbreviation “DLCL” refers to poly(DL-lactide-co-caprolactone).

The abbreviation “LCL” refers to poly(L-lactide-co-caprolactone).

The abbreviation “G” refers to polyglycolide.

The abbreviation “PEG” refers to poly(ethylene glycol).

The abbreviation “PLGA” refers to poly(lactide-co-glycolide) also known as poly(lactic-co-glycolic acid), which are used interchangeably.

The abbreviation “PLA” refers to polylactide.

The abbreviation “POE” refers to poly(orthoester).

In one embodiment, an implantable drug depot is provided that is useful for reducing, preventing or treating pain and/or inflammation in a patient in need of such treatment, the implantable drug depot being implantable at a site beneath the skin and comprising an effective amount of an analgesic and/or an anti-inflammatory agent disposed within a reversible phase transition material of the drug depot, wherein the reversible phase transition material is capable of releasing a bolus dose of the analgesic, muscle relaxant and/or the anti-inflammatory agent when heat, cold or another suitable form of energy, e.g., ultrasound energy, light, mechanical energy (such as agitation, massage, etc.) electrical, chemical, or magnetic energy is applied to the skin of a patient to reduce, prevent or treat pain and/or inflammation.

In one embodiment, the analgesic, muscle relaxant and/or the anti-inflammatory agent can be used as a racemic mixture. In yet another embodiment, the analgesic, muscle relaxant and/or the anti-inflammatory agent is used as a single stereoisomer. In another embodiment, the analgesic, muscle relaxant and/or the anti-inflammatory agent is used as a mixture of stereo isomers containing equal (1:1) or unequal amounts of stereoisomers. For example, in some embodiments, the analgesic, muscle relaxant and/or the anti-inflammatory agent may comprise mixtures of (+)R and (−) enantiomers. In various embodiments, the analgesic, muscle relaxant and/or the anti-inflammatory agent may comprise a 1:1 racemic mixture of the analgesic, muscle relaxant and/or the anti-inflammatory agent.

The target tissue site chosen for analgesic, muscle relaxant and/or the anti-inflammatory agent delivery depends on, among other things, upon the condition being treated, desired therapeutic concentration of the drug to be achieved in the patient and the duration of drug concentration that must be maintained.

In some embodiments, local administration of the drug depot at or near the target tissue site allows for a lower dose of the analgesic, muscle relaxant and/or the anti-inflammatory agent to be used than the usual oral, intravenous, or intramuscular dose. For example, local administration of the drug depot can be accomplished with daily doses that are 50%, 45%, 40%, 35%, 30%, 25%, 20%, 15%, 10%, 5%, 1%, 0.5%, 0.1%, 0.01% of the usual oral, intravenous or intramuscular dose. In turn, systemic side effects, such as for example, liver transaminase elevations, hepatitis, liver failure, myopathy, constipation, etc. may be reduced or eliminated.

The concentration of analgesic and/or anti-inflammatory agent included in the drug depot and used in the methodologies described herein is a concentration effective to produce a therapeutic effect of preventing, treating or reducing pain and/or inflammation. Dosages of analgesic and/or anti-inflammatory agent, e.g., clonidine for producing an analgesic effect in human patients upon local administration can typically range in some embodiments from between about 150 micrograms to 800 micrograms per day or from 3-12 micrograms/hour by local infusion.

However, as will be understood by the skilled artisan upon reading this disclosure, the effective concentration will vary depending upon the analgesic and/or anti-inflammatory agent selected, the route of administration, the frequency of administration, the formulation administered, and the condition being treated.

In one embodiment, the analgesic, muscle relaxant and/or the anti-inflammatory agent is administered in an amount of about 0.0001 mg/kg/day to about 40 mg/kg/day for reducing, preventing or treating pain and/or inflammation that follows, for example, surgery, chronic inflammatory diseases, chronic pelvic pain syndromes (e.g., interstitial cystitis, chronic non-bacterial prostatitis, vulvodynia, endometriosis, irritable bowel disease and other conditions that result in chronic pain in the pelvic region), bursitis, osteoarthritis, osteolysis, tendonitis, sciatica, herniated discs, stenosis, myopathy, spondilothesis, lower back pain, facet pain, carpal tunnel syndrome, tarsal tunnel syndrome, failed back pain or the like. In another embodiment, the analgesic, muscle relaxant and/or the anti-inflammatory agent is administered in an amount of about 0.001 mg/kg/day to about 4 mg/kg/day. In one embodiment, the analgesic, muscle relaxant and/or the anti-inflammatory agent is administered in an amount of about 0.01 mg/kg/day to about 0.4 mg/kg/day.

Analgesic and/or Anti-Inflammatory Agent

Analgesic refers to an agent or compound that can reduce, relieve or eliminate pain. Examples of analgesic agents include but are not limited to acetaminophen, a local anesthetic, such as for example, lidocaine, bupivacaine, benzocaine, ropivacaine, clonidine, amitriptyline, carbamazepine, gabapentin, pregabalin, opioid analgesics or a combination thereof. Particular anesthetics include by way of example and not limitation, aliflurane; baclofen, benoxinate hydrochloride; benzocaine; biphenamine hydrochloride; bupivacaine hydrochloride; butamben; butamben picrate; clonidine, clonidine hydrochloride, chloroprocaine hydrochloride; cocaine; cocaine hydrochloride; cyclopropane; desflurane; dexivacaine; diamocaine cyclamate; dibucaine; dibucaine hydrochloride; dyclonine hydrochloride; enflurane; ether; ethyl chloride; etidocaine; etoxadrol hydrochloride; euprocin hydrochloride; fluroxene; halothane; isobutamben; isoflurane; ketamine hydrochloride; levoxadrol hydrochloride; lidocaine; lidocaine hydrochloride; mepivacaine hydrochloride; methohexital sodium; methoxyflurane; midazolam hydrochloride; midazolam maleate; minaxolone; nitrous oxide; norflurane; octodrine; oxethazaine; phencyclidine hydrochloride; pramoxine hydrochloride; prilocaine hydrochloride; procaine hydrochloride; propanidid; proparacaine hydrochloride; propofol; propoxycaine hydrochloride; pyrrocaine; risocaine; rodocaine; roflurane; salicyl alcohol; sevoflurane; teflurane; tetracaine; tetracaine hydrochloride; thiamylal; thiamylal sodium; thiopental sodium; tiletamine hydrochloride; zolamine hydrochloride; or combinations thereof.

Opioid analgesics include, alfentanil, allylprodine, alphaprodine, anileridine, benzylmorphine, bezitramide, buprenorphine, butorphanol, clonitazene, codeine, desomorphine, dextromoramide, dextropropoxyphene, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, flupirtine, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pethidine, pentazocine, phenadoxone, phenomorphan, phenazocine, phenoperidine, piminodine, piritramide, propheptazine, promedol, properidine, propoxyphene, remifentanil, sufentanil, tilidine, tramadol or a combination thereof.

The phrase “anti-inflammatory agent” refers to an agent or compound that has anti-inflammatory effects. These agents may remedy pain by reducing inflammation. Examples of anti-inflammatory agents include, but are not limited to, a statin, sulindac, sulfasalazine, guanidinoethyldisulfide, naroxyn, diclofenac, indomethacin, ibuprofen, flurbiprofen, ketoprofen, aclofenac, aloxiprin, aproxen, aspirin, diflunisal, fenoprofen, mefenamic acid, naproxen, phenylbutazone, piroxicam, meloxicam, salicylamide, salicylic acid, desoxysulindac, tenoxicam, ketoralac, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, triflumidate, fenamates (mefenamic acid, meclofenamic acid), nabumetone, celecoxib, etodolac, nimesulide, apazone, gold, tepoxalin; dithiocarbamate, or a combination thereof. Anti-inflammatory agents also include other compounds such as steroids, such as for example, fluocinolone, cortisol, cortisone, hydrocortisone, fludrocortisone, prednisone, prednisolone, methylprednisolone, triamcinolone, betamethasone, dexamethasone, beclomethasone, fluocinolone, fluticasone interleukin-1 receptor antagonists, thalidomide (a TNF-α release inhibitor), thalidomide analogues (which reduce TNF-α production by macrophages), bone morphogenetic protein (BMP) type 2 or BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α), guanidinoethyldisulfide, or a combination thereof.

Exemplary anti-inflammatory agents include, for example, naproxen; diclofenac; celecoxib; sulindac; diflunisal; piroxicam; indomethacin; etodolac; meloxicam; ibuprofen; ketoprofen; r-flurbiprofen; mefenamic; nabumetone; sulfasalazine, sulindac, tolmetin, and sodium salts of each of the foregoing; ketorolac bromethamine; ketorolac tromethamine; ketorolac acid; choline magnesium trisalicylate; rofecoxib; valdecoxib; lumiracoxib; etoricoxib; aspirin; salicylic acid and its sodium salt; salicylate esters of alpha, beta, gamma-tocopherols and tocotrienols (and all their d, 1, and racemic isomers); methyl, ethyl, propyl, isopropyl, n-butyl, sec-butyl, t-butyl, esters of acetylsalicylic acid; tenoxicam; aceclofenac; nimesulide; nepafenac; amfenac; bromfenac; flufenamate; phenylbutazone, or a combination thereof.

Exemplary steroids that are considered anti-inflammatory agents include, for example, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, dexamethasone 21-acetate, dexamethasone 21-phosphate di-Na salt, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide or a combination thereof.

Examples of a useful statin for treatment of pain and/or inflammation include, but is not limited to, atorvastatin, simvastatin, pravastatin, cerivastatin, mevastatin (see U.S. Pat. No. 3,883,140, the entire disclosure is herein incorporated by reference), velostatin (also called synvinolin; see U.S. Pat. Nos. 4,448,784 and 4,450,171 these entire disclosures are herein incorporated by reference), fluvastatin, lovastatin, rosuvastatin and fluindostatin (Sandoz XU-62-320), dalvastain (EP Appln. Publn. No. 738510 A2, the entire disclosure is herein incorporated by reference), eptastatin, pitavastatin, or pharmaceutically acceptable salts thereof or a combination thereof. In various embodiments, the statin may comprise mixtures of (+)R and (−)-S enantiomers of the statin. In various embodiments, the statin may comprise a 1:1 racemic mixture of the statin.

Anti-inflammatory agents also include those with anti-inflammatory properties, such as, for example, amitriptyline, carbamazepine, gabapentin, pregabalin, clonidine, or a combination thereof.

Unless otherwise specified or apparent from context, where this specification and the set of claims that follows refer to an analgesic, muscle relaxant, and/or anti-inflammatory agent, the inventor is also referring to a pharmaceutically acceptable salt of the analgesic and/or anti-inflammatory agent including stereoisomers. Pharmaceutically acceptable salts include those salt-forming acids and bases that do not substantially increase the toxicity of the compound. Some examples of potentially suitable salts include salts of alkali metals such as magnesium, calcium, sodium, potassium and ammonium, salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, or the like.

A “drug depot” is the composition in which at least one analgesic and/or anti-inflammatory agent is administered to the body. Thus, a drug depot may comprise a physical structure to facilitate implantation and retention in a desired site (e.g., a disc space, a spinal canal, a tissue of the patient, particularly at or near a site of surgery, or other site of inflammation, etc.). The drug depot also comprises the drug itself. The term “drug” as used herein is generally meant to refer to any substance that alters the physiology of a patient. The term “drug” may be used interchangeably herein with the terms “therapeutic agent,” “therapeutically effective amount,” and “active pharmaceutical ingredient” or “API.” It will be understood that unless otherwise specified a “drug” formulation may include more than one therapeutic agent, wherein exemplary combinations of therapeutic agents include a combination of two or more drugs. The drug provides a concentration gradient of the therapeutic agent for delivery to the site. In various embodiments, the drug depot provides an optimal drug concentration gradient of the therapeutic agent at a distance of up to about 0.1 mm to about 5 cm from the implant site, and comprises at least one analgesic and/or anti-inflammatory agent or its pharmaceutically acceptable salt.

A “depot” includes but is not limited to capsules, microspheres, microparticles, microcapsules, microfibers particles, nanospheres, nanoparticles, coating, matrices, wafers, pills, pellets, emulsions, ointments, liposomes, micelles, gels, fiber, strip, sheet or other pharmaceutical delivery compositions or a combination thereof. In some embodiments, the drug depot has pores that allow release of the drug from the depot. The drug depot will allow fluid in the depot to displace the drug. However, cell infiltration into the depot will be prevented by the size of the pores of the depot. In this way, in some embodiments, the depot should not function as a tissue scaffold and allow tissue growth. Rather, the drug depot will solely be utilized for drug delivery. In some embodiments, the pores in the drug depot will be less than 10-50 microns. This pore size will prevent cells from infiltrating the drug depot and laying down scaffolding cells. Thus, in this embodiment, drug will elute from the drug depot as fluid enters the drug depot, but cells will be prevented from entering. In some embodiments, where there are little or no pores, the drug will elute out from the drug depot by the action of enzymes, by hydrolytic action, diffusion and/or by other similar mechanisms in the human body.

Suitable materials for the depot are ideally pharmaceutically acceptable biodegradable and/or any bioabsorbable materials that are preferably FDA approved or GRAS materials. These materials can be polymeric or non-polymeric, as well as synthetic or naturally occurring, or a combination thereof. In various embodiments, the drug depot may not be biodegradable or comprise material that is not biodegradable. Non-biodegradable polymers include, but are not limited to, various cellulose derivatives (carboxymethyl cellulose, cellulose acetate, cellulose acetate propionate, ethyl cellulose, hydroxypropyl methyl cellulose, hydroxyalkyl methyl celluloses, and alkyl celluloses), silicon and silicon-based polymers (such as polydimethylsiloxane), polyethylene-co-(vinyl acetate), poloxamer, polyvinylpyrrolidone, poloxamine, polypropylene, polyamide, polyacetal, polyurethane, poly(ester-amide), polyester, poly ethylene-chlorotrifluoroethylene, polytetrafluoroethylene (PTFE or “Teflon™”), styrene butadiene rubber, polyethylene, polypropylene, polyphenylene oxide-polystyrene, poly-α-chloro-p-xylene, polymethylpentene, polysulfone, non-degradable ethylene-vinyl acetate (e.g., ethylene vinyl acetate disks and poly(ethylene-co-vinyl acetate)), and other related biostable polymers or combinations thereof. Suitable drug depots for use in the present application are described in U.S. Provisional Application No. 61/046,246 filed Apr. 18, 2008, U.S. Provisional Application No. 61/046,218 filed Apr. 18, 2008, U.S. Provisional Application No. 61/046,218 filed Apr. 18, 2008, U.S. Provisional Application No. 61/046,201 filed Apr. 18, 2008, U.S. Ser. No. 12/105,864 filed Apr. 18, 2008 and U.S. Ser. No. 12/105,375 filed Apr. 18, 2008. The entire disclosures of these applications are herein incorporated by reference into the present application.

The drug depot may comprise non-resorbable polymers as well. These non-resorbable polymers can include, but are not limited to, delrin, polyurethane, copolymers of silicone and polyurethane, polyolefins (such as polyisobutylene and polyisoprene), acrylamides (such as polyacrylic acid and poly(acrylonitrile-acrylic acid)), neoprene, nitrile, acrylates (such as polyacrylates, poly(2-hydroxy ethyl methacrylate), methyl methacrylate, 2-hydroxyethyl methacrylate, and copolymers of acrylates with N-vinyl pyrrolidone), N-vinyl lactams, polyacrylonitrile, glucomannan gel, vulcanized rubber and combinations thereof. Examples of polyurethanes include thermoplastic polyurethanes, aliphatic polyurethanes, segmented polyurethanes, hydrophilic polyurethanes, polyether-urethane, polycarbonate-urethane and silicone polyether-urethane. Typically, the non-degradable drug depots may need to be removed.

“Reversible phase transition material” includes material that changes phases or physical state (e.g., solid to liquid, solid to semi-solid, semi-solid to liquid, liquid to solid, liquid to semi-solid, or semi-solid to solid, glass-rubber, crystalline or semi-crystalline to melt, water-insoluble to water soluble) in response to an external stimuli, such as for example, change in temperature. The intended effect of changing the phase or physical state of the reversible phase transition material is to increase the rate of drug permeation within the drug depot to increase the release rate of the drug from the depot. Reversible means that the phase change material returns toward its initial phase or physical state at some time after removal of the external stimuli. The material can include non-biodegradable or biodegradable polymeric and non-polymeric material. A “therapeutically effective amount” or “effective amount” is such that when administered, the drug results in alteration of the biological activity, such as, for example, inhibition of inflammation, reduction or alleviation of pain, improvement in the disease and/or condition being treated, etc. The dosage administered to a patient can unless otherwise specified or apparent from context be as single or multiple doses depending upon a variety of factors, including the drug's administered pharmacokinetic properties, the route of administration, patient conditions and characteristics (sex, age, body weight, health, size, etc.), extent of symptoms, concurrent treatments, frequency of treatment and the effect desired. In some embodiments the formulation is designed for immediate release upon application of heat, cold or another suitable form of energy, e.g., ultrasound energy, light, mechanical energy (such as agitation), electrical, chemical, or magnetic energy. In other embodiments the formulation is designed for sustained release. In other embodiments, the formulation comprises one or more immediate release surfaces or layers and one or more sustain release surfaces or layers.

The phrases “sustained release” or “sustain release” (also referred to as extended release or controlled release) are used herein to refer to one or more therapeutic agent(s) that is introduced into the body of a human or other mammal and continuously or continually releases a stream of one or more therapeutic agents over a predetermined time period and at a therapeutic level sufficient to achieve a desired therapeutic effect throughout the predetermined time period. Reference to a continuous or continual release stream is intended to encompass release that occurs as the result of biodegradation in vivo of the drug depot or component thereof, or as the result of metabolic transformation or dissolution of the therapeutic agent(s) or conjugates of therapeutic agent(s). As persons of ordinary skill are aware, sustained release formulations may, by way of example, be created as films, slabs, sheets, pellets, microparticles, microspheres, microcapsules, spheroids, shaped derivatives or paste. The formulations may be in a form that is suitable for suspension in isotonic saline, physiological buffer or other solution acceptable for injection into a patient. Further, the formulations may be used in conjunction with any implantable, insertable or injectable system that a person of ordinary skill would appreciate as useful in connection with embodiments herein including but not limited to parenteral formulations, microspheres, microcapsules, gels, pastes, ointments, creams, implantable rods, pellets, plates or fibers, etc. In some embodiments, the drug depot comprises material (e.g., polymers) that causes sustained release of the analgesic and/or anti-inflammatory agent.

The phrase “immediate release” is used herein to refer to one or more therapeutic agent(s) that is introduced into the body and that is allowed to dissolve in or become absorbed at the location to which it is administered, with no intention of delaying or prolonging the dissolution or absorption of the drug. Immediate release refers to the release of drug within a short time period following administration, e.g., generally within a few minutes to about 1 hour. In some embodiments, the drug depot has the analgesic and/or anti-inflammatory agent disposed within it to provide an immediate release of the analgesic and/or anti-inflammatory agent. For example, in some embodiments, the drug depot may comprise a reversible phase transition polymer that changes phase or physical state upon application of heat, cold or another suitable form of energy to the depot or near the depot (e.g., skin above where the drug depot has been implanted) to cause an increased rate of release of the analgesic and/or anti-inflammatory agent.

The term “mammal” refers to organisms from the taxonomy class “mammalian,” including but not limited to humans, other primates such as chimpanzees, apes, orangutans and monkeys, rats, mice, cats, dogs, cows, horses, etc. In various embodiments, the mammal is a human patient.

The phrase “release rate profile” refers to the percentage of active ingredient that is released over fixed units of time, e.g., mcg/hr, mcg/day, mg/hr, mg/day, 10% per day for ten days, etc. As persons of ordinary skill know, a release rate profile may be but need not be linear. By way of a non-limiting example, the drug depot may be a pellet that releases at least one alpha adrenergic receptor agonist over a period of time.

Treating or treatment of a disease or condition refers to executing a protocol, which may include administering one or more drugs to a patient (human, normal or otherwise, or other mammal), in an effort to alleviate signs or symptoms of the condition/disease. Alleviation can occur prior to signs or symptoms of the disease or condition appearing, as well as after their appearance. Thus, “treating” or “treatment” includes “preventing” or “prevention” of disease or undesirable condition. In addition, “treating” or “treatment” does not require complete alleviation of signs or symptoms, does not require a cure, and specifically includes protocols that have only a marginal effect on the patient. “Reducing pain and/or inflammation” includes a decrease in pain and/or inflammation and does not require complete alleviation of pain and/or inflammation signs or symptoms, and does not require a cure. In various embodiments, reducing pain and/or inflammation includes even a marginal decrease in pain and/or inflammation. By way of example, the administration of the effective dosage analgesic and/or anti-inflammatory agent may be used to prevent, treat or relieve the symptoms of pain and/or inflammation for different diseases or conditions. These disease/conditions may comprise post-operative pain and/or inflammation, bursitis, tendonitis, chronic inflammatory diseases, including, but not limited to autoimmune diseases, such as multiple sclerosis, rheumatoid arthritis, osteoarthritis, insulin dependent diabetes (type I diabetes), systemic lupus erythrematosis and psoriasis, immune pathologies induced by infectious agents, such as helminthic (e.g., leishmaniasis) and certain viral infections, including HIV, and bacterial infections, including Lyme disease, tuberculosis and lepromatous leprosy, tissue transplant rejection, graft versus host disease and atopic conditions, such as asthma and allergy, including allergic rhinitis, gastrointestinal allergies, including food allergies, eosinophilia, conjunctivitis or glomerular nephritis.

One chronic condition is sciatica. In general, sciatica” is an example of pain that can transition from nociceptive to neuropathic pain. Sciatica refers to pain associated with the sciatic nerve which runs from the lower part of the spinal cord (the lumbar region), down the back of the leg and to the foot. Sciatica generally begins with a herniated disc. The herniated disc itself leads to local immune system activation. The herniated disc also may damage the nerve root by pinching or compressing it, leading to additional immune system activation in the area. In various embodiments, the analgesic and/or anti-inflammatory agent may be used to reduce, treat, or prevent sciatic pain and/or inflammation by locally administering the analgesic and/or anti-inflammatory agent at one or more target tissue sites (e.g., nerve root, dorsal root ganglion, focal sites of pain, at or near the spinal column, etc.).

“Localized” delivery includes delivery where one or more drugs are deposited within a tissue, for example, a nerve root of the nervous system or a region of the brain, or in close proximity (within about 10 cm, or within about 5 cm, or within 0.1 cm for example) thereto. A “targeted delivery system” provides delivery of one or more drugs depots, gels or depot dispersed in the gel having a quantity of therapeutic agent that can be deposited at or near the target site as needed for treatment of pain, inflammation or other disease or condition.

The term “biodegradable” includes that all or parts of the drug depot will degrade over time by the action of enzymes, by hydrolytic action and/or by other similar mechanisms in the human body. In various embodiments, “biodegradable” includes that the depot (e.g., microparticle, microsphere, etc.) can break down or degrade within the body to non-toxic components after or while a therapeutic agent has been or is being released. By “bioerodible” it is meant that the depot will erode or degrade over time due, at least in part, to contact with substances found in the surrounding tissue, fluids or by cellular action. By “bioabsorbable” it is meant that the depot will be broken down and absorbed within the human body, for example, by a cell or tissue. “Biocompatible” means that the depot will not cause substantial tissue irritation or necrosis at the target tissue site.

The phrase “pain management medication” includes one or more therapeutic agents that are administered to prevent, alleviate or remove pain entirely. These include one or more analgesic agents and/or anti-inflammatory agents alone or in combination with, muscle relaxants, anesthetics, or so forth, or combinations thereof.

In various embodiments, the depot can be designed to cause a burst dose or bolus dose of therapeutic agent within the first 24 hours to 48 hours after implantation. “Initial burst” or “burst effect” or “bolus dose” refers to the immediate release of a dose of the therapeutic agent from the depot within 5, 10, 15, 20, 25, 30, 25, 40, 45, 50, 55, 60, 75, 90, 100, 120, 140, 160, 180 minutes or within 4-6 hours. In some embodiments, the bolus dose would occur within a few minutes to within an hour after the depot comes in contact with heat, cold or other suitable form of energy, e.g., ultrasound energy, light, mechanical energy (such as agitation), electrical, chemical, or magnetic energy and an aqueous fluid (e.g., blood, synovial fluid, cerebral spinal fluid, etc.). This burst effect or bolus dose is particularly beneficial for the analgesic and/or anti-inflammatory, where breakthrough pain and/or inflammation is experienced by the patient. In some embodiments, by the application of heat, cold or another suitable form of energy, e.g., ultrasound energy, light, mechanical energy (such as agitation), electrical, chemical, or magnetic energy, the patient can control the bolus dosing and their analgesia. The “burst effect” or “bolus dose” is believed to be due to the increased release of therapeutic agent from the depot. In some embodiments, the drug depot has the analgesic and/or anti-inflammatory agent disposed within it to provide an immediate release of the analgesic and/or anti-inflammatory agent. For example, in some embodiments, the drug depot may comprise a reversible phase transition polymer that changes phases upon application of heat and/or cold to the depot or near the depot (e.g., within 5, 4, 3, 2, 1, 0.5, 0.1, 0.01 cm of it) to cause release of a bolus dose of the analgesic and/or anti-inflammatory agent. Typically, the bolus dose can be 10, 20, 30, 40, 50, 60, 70, 80, 90, or 100% of the locally administered daily dose, however, this dose is released within 5, 10, 15, 20, 25, 30, 25, 40, 45, 50, 55, 60, 75, 90, 100, 120, 140, 160, 180 minutes or within 4-6 hours, instead of within 24 to 48 hours. For example, if the drug depot is designed to release 100 μg/day of an alpha agonist (e.g., clonidine), the burst effect or bolus dose will allow the drug depot to release 100 μg of the alpha agonist within 5, 10, 15, 20, 25, 30, 25, 40, 45, 50, 55, 60, 75, 90, 100, 120, 140, 160, or 180 minutes to relieve pain and/or inflammation.

The drug depot comprising at least one analgesic and/or anti-inflammatory agent or its pharmaceutically acceptable salt may be co-administered with a muscle relaxant. Co-administration may involve administering at the same time in separate drug depots or formulating together in the same drug depot.

Exemplary muscle relaxants include by way of example and not limitation, alcuronium chloride, atracurium bescylate, baclofen, carbolonium, carisoprodol, chlorphenesin carbamate, chlorzoxazone, cyclobenzaprine, dantrolene, decamethonium bromide, fazadinium, gallamine triethiodide, hexafluorenium, meladrazine, mephensin, metaxalone, methocarbamol, metocurine iodide, pancuronium, pridinol mesylate, styramate, suxamethonium, suxethonium, thiocolchicoside, tizanidine, tolperisone, tubocuarine, vecuronium, or combinations thereof.

The drug depot may also comprise other therapeutic agents or active ingredients in addition to in place of the analgesic and/or anti-inflammatory agent or its pharmaceutically acceptable salt. Suitable additional therapeutic agents include, but are not limited to, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibodies (daclizumab, basilicimab), ABX (anti IL-8 antibodies), recombinant human IL-10, or HuMax IL-15 (anti-IL 15 antibodies).

Other suitable therapeutic agents that may be co-administered with the alpha adrenergic agonist include IL-1 inhibitors, such Kineret® (anakinra) which is a recombinant, non-glycosylated form of the human interleukin-1 receptor antagonist (IL-1Ra), or AMG 108, which is a monoclonal antibody that blocks the action of IL-1. Therapeutic agents also include excitatory amino acids such as glutamate and aspartate, antagonists or inhibitors of glutamate binding to NMDA receptors, AMPA receptors, and/or kainate receptors. It is contemplated that where desirable a pegylated form of the above may be used. Examples of other therapeutic agents include NF kappa B inhibitors such as glucocorticoids, or antioxidants, such as dilhiocarbamate.

Specific examples of additional therapeutic agents suitable for use include, but are not limited to, an anabolic growth factor or anti-catabolic growth factor, or an osteoinductive growth factor or a combination thereof.

Suitable anabolic growth or anti-catabolic growth factors include, but are not limited to, a bone morphogenetic protein, a growth differentiation factor, a LIM mineralization protein, CDMP or progenitor cells or a combination thereof.

For each analgesic and/or anti-inflammatory agent, in some embodiments, particular when the agent(s) is disposed in the biodegradable polymer layer, which provides sustained release properties to the drug depot, the release of each compound may be for at least one, at least two, at least three, at least four, at least five, at least six, at least seven, at least eight, at least nine, at least ten, at least eleven, at least twelve, at least thirteen, at least fourteen, or at least fifteen days, or longer. In some embodiments, the drug depot provides relief of post-operative pain and/or inflammation for about 3 days to about 10 days.

The therapeutic agent (e.g., analgesic and/or anti-inflammatory agent) also includes its pharmaceutically acceptable salt. As used herein, “pharmaceutically acceptable salts” refer to derivatives of the disclosed compounds (including, for example, esters or amines) wherein the parent compound may be modified by making acidic or basic salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, or nitric acids; or the salts prepared from organic acids such as acetic, fuoric, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, isethionic acid. Pharmaceutically acceptable also includes the racemic mixtures ((+)-R and (−)-S enantiomers) or each of the dextro and levo isomers of the therapeutic agent individually. The therapeutic agent may be in the free acid or base form or be pegylated for long acting activity.

### Clonidine

In one embodiment, the analgesic and/or anti-inflammatory comprises clonidine. When referring to clonidine, unless otherwise specified or apparent from context it is understood that the inventor is also referring to pharmaceutically acceptable salts. One well-known commercially available salt for clonidine is its hydrochloride salt. Some other examples of potentially pharmaceutically acceptable salts include those salt-forming acids and bases that do not substantially increase the toxicity of a compound, such as, salts of alkali metals such as magnesium, potassium and ammonium, salts of mineral acids such as hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, and the like.

Further, when referring to clonidine the active ingredient may not only be in the salt form, but also in the base form (e.g., free base). In various embodiments, if it is in the base form, it may be combined with polymers under conditions in which there is not severe polymer degradation, as may be seen upon heat or solvent processing that may occur with PLGA or PLA. By way of a non limiting example, when formulating clonidine with poly(orthoesters) it may be desirable to use the clonidine base formulation. By contrast, when formulating clonidine with PLGA, it may be desirable to use the HCl salt form.

In one embodiment, the drug depot comprises clonidine, also referred to as 2,6-dichloro-N-2-imidazolidinyldenebenzenamine. Clonidine or a pharmaceutically acceptable salt thereof is available from various pharmaceutical manufactures for reducing, preventing or treating pain and/or inflammation that follows, for example, surgery, chronic inflammatory diseases, chronic inflammatory bowel disease, bursitis, osteoarthritis, osteolysis, tendonitis, sciatica, herniated discs, stenosis, myopathy, spondilothesis, lower back pain, facet pain, carpal tunnel syndrome, tarsal tunnel syndrome, failed back pain or the like.

The dosage may be from approximately 0.0005 to approximately 960 μg/day. Additional dosages of clonidine include from approximately 0.0005 to approximately 900 μg/day; approximately 0.0005 to approximately 500 μg/day; approximately 0.0005 to approximately 250 μg/day; approximately 0.0005 to approximately 100 μg/day; approximately 0.0005 to approximately 75 μg/day; approximately 0.001 to approximately 70 μg/day; approximately 0.001 to approximately 65 μg/day; approximately 0.001 to approximately 60 μg/day; approximately 0.001 to approximately 55 μg/day; approximately 0.001 to approximately 50 μg/day; approximately 0.001 to approximately 45 μg/day; approximately 0.001 to approximately 40 μg/day; approximately 0.001 to approximately 35 μg/day; approximately 0.0025 to approximately 30 μg/day; approximately 0.0025 to approximately 25 μg/day; approximately 0.0025 to approximately 20 μg/day; approximately 0.0025 to approximately 15 μg/day; approximately 0.0025 to approximately 10 μg/day; approximately 0.0025 to approximately 5 μg/day; and approximately 0.0025 to approximately 2.5 μg/day. In another embodiment, the dosage of clonidine is from approximately 0.005 to approximately 15 μg/day. In another embodiment, the dosage of clonidine is from approximately 0.005 to approximately 10 μg/day. In another embodiment, the dosage of clonidine is from approximately 0.005 to approximately 5 μg/day. In another embodiment, the dosage of clonidine is from approximately 0.005 to 2.5 μg/day. In some embodiments, the amount of clonidine is between 40 and 600 μg/day. In some embodiments, the amount of clonidine is between 200 and 400 μg/day.

In various embodiments, there is a pharmaceutical formulation comprising: clonidine, wherein the clonidine comprises from about 1 wt. % to about 20 wt. % of the formulation, and at least one biodegradable polymer. In some embodiments, the clonidine comprises from about 3 wt. % to about 20 wt. %, about 3 wt. % to about 18 wt. %, about 5 wt. % to about 15 wt. % or about 7.5 wt. % to about 12.5 wt. % of the formulation. By way of example, when using a 5%-15% clonidine composition, the mole ratio of clonidine to polymer would be from approximately 16-52 when using an approximately 80 kDalton polymer that has a 267 grams/mole ratio.

In some embodiments, the at least one biodegradable polymer comprises poly(lactic-co-glycolide) (PLGA) or poly(orthoester) (POE) or a combination thereof. The poly(lactic-co-glycolide) may comprise a mixture of polyglycolide (PGA) and polylactide and in some embodiments, in the mixture, there is more polylactide than polyglycolide. In various embodiments there is 100% polylactide and 0% polyglycolide; 95% polylactide and 5% polyglycolide; 90% polylactide and 10% polyglycolide; 85% polylactide and 15% polyglycolide; 80% polylactide and 20% polyglycolide; 75% polylactide and 25% polyglycolide; 70% polylactide and 30% polyglycolide; 65% polylactide and 35% polyglycolide; 60% polylactide and 40% polyglycolide; 55% polylactide and 45% polyglycolide; 50% polylactide and 50% polyglycolide; 45% polylactide and 55% polyglycolide; 40% polylactide and 60% polyglycolide; 35% polylactide and 65% polyglycolide; 30% polylactide and 70% polyglycolide; 25% polylactide and 75% polyglycolide; 20% polylactide and 80% polyglycolide; 15% polylactide and 85% polyglycolide; 10% polylactide and 90% polyglycolide; 5% polylactide and 95% polyglycolide; and 0% polylactide and 100% polyglycolide.

In various embodiments that comprise both polylactide and polyglycolide; there is at least 95% polylactide; at least 90% polylactide; at least 85% polylactide; at least 80% polylactide; at least 75% polylactide; at least 70% polylactide; at least 65% polylactide; at least 60% polylactide; at least 55%; at least 50% polylactide; at least 45% polylactide; at least 40% polylactide; at least 35% polylactide; at least 30% polylactide; at least 25% polylactide; at least 20% polylactide; at least 15% polylactide; at least 10% polylactide; or at least 5% polylactide; and the remainder of the biopolymer is polyglycolide.

In various embodiments, the drug particle size used in the drug depot is from about 5 to 30 micrometers, however, in various embodiments ranges from about 1 micron to 250 microns may be used. In some embodiments, the biodegradable polymer comprises at least 50 wt. %, at least 60 wt. %, at least 70 wt. %, at least 80 wt. % of the formulation, at least 85 wt. % of the formulation, at least 90 wt. % of the formulation, at least 95 wt. % of the formulation or at least 97 wt. % of the formulation. In some embodiments, the at least one biodegradable polymer and the clonidine are the only components of the pharmaceutical formulation.

In some embodiments, at least 75% of the particles have a size from about 10 micrometer to about 200 micrometers. In some embodiments, at least 85% of the particles have a size from about 10 micrometer to about 200 micrometers. In some embodiments, at least 95% of the particles have a size from about 10 micrometer to about 200 micrometers. In some embodiments, all of the particles have a size from about 10 micrometer to about 200 micrometers.

In some embodiments, at least 75% of the particles have a size from about 20 micrometer to about 180 micrometers. In some embodiments, at least 85% of the particles have a size from about 20 micrometers to about 180 micrometers. In some embodiments, at least 95% of the particles have a size from about 20 micrometer to about 180 micrometers. In some embodiments, all of the particles have a size from about 20 micrometer to about 180 micrometers.

In some embodiments, there is a pharmaceutical formulation in a drug depot comprising: clonidine, wherein the clonidine is in the form of a hydrochloride salt, and comprises from about 1 wt. % to about 20 wt. % of the formulation, and at least one biodegradable polymer, wherein the at least one biodegradable polymer comprises poly(lactide-co-glycolide) (or poly(lactic-co-glycolic acid)) or poly(orthoester) or a combination thereof, and said at least one biodegradable polymer comprises at least 80 wt. % of said formulation.

In some embodiments, there are methods for treating acute pain. These methods comprise: administering a pharmaceutical composition to an organism, wherein said pharmaceutical composition comprises from about 1 wt. % to about 20 wt. % of the formulation, and at least one biodegradable polymer. In some embodiments, the loading is from about 5 wt. % to about 10 wt. %. In some embodiments, the loading is from about 10 wt. % to about 20 wt. %.

In some embodiment there is a higher loading of clonidine, e.g., at least 20 wt. %, at least 30 wt. %, at least 40 wt. %, at least 50 wt. %, at least 60 wt. %, at least 70 wt. %, at least 80 wt. %, or at least 90 wt. %.

In some embodiments, the drug depot contains excipients along with the clonidine. Exemplary excipients that may be formulated with clonidine in addition to the biodegradable polymer include but are not limited to MgO (e.g., 1 wt. %), 5050 DLG 6E, 5050 DLG 1A, mPEG, TBO-Ac, mPEG, Span-65, Span-85, pluronic F127, TBO-Ac, sorbital, cyclodextrin, maltodextrin, pluronic F68, CaCl, 5050 7A and combinations thereof. In some embodiments, the excipients comprise from about 0.001 wt. % to about 50 wt. % of the formulation. In some embodiments, the excipients comprise from about 0.001 wt. % to about 40 wt. % of the formulation. In some embodiments, the excipients comprise from about 0.001 wt. % to about 30 wt. % of the formulation. In some embodiments, the excipients comprise from about 0.001 wt. % to about 20 wt. % of the formulation. In some embodiments, the excipients comprise from about 0.001 wt. % to about 10 wt. % of the formulation. In some embodiments, the excipients comprise from about 0.001 wt. % to about 50 wt. % of the formulation. In some embodiments, the excipients comprise from about 0.001 wt. % to about 2 wt. % of the formulation.

A strategy of triangulation may be effective when administering these pharmaceutical formulations. Thus, a plurality (at least two, at least three, at least four, at least five, at least six, at least seven, etc.) drug depots comprising the pharmaceutical formulations may be placed around the target tissue site (also known as the pain generator or pain generation site) such that the target tissue site falls within a region that is either between the formulations when there are two, or within an area whose perimeter is defined by a set of plurality of formulations.

In some embodiments, the formulations are slightly rigid with varying length, widths, diameters, etc. For example, certain formulations may have a diameter of 0.50 mm and a length of 4 mm. It should be noted that particle size may be altered by techniques such as using a mortar and pestle, jet-drying or jet milling.

In some embodiments, clonidine is released at a rate of 2-3 μg per day for a period of at least three days. In some embodiments, this release rate continues for, at least ten days, at least fifteen days, at least twenty-five days, at least fifty days, at least ninety days, at least one hundred days, at least one-hundred and thirty-five days, at least one-hundred and fifty days, or at least one hundred and eighty days. For some embodiments, 300-425 micrograms of clonidine as formulated with a biopolymer are implanted into a person at or near a target tissue site. If clonidine is implanted at multiple sites that triangulate the target site then in some embodiments, the total amount of clonidine at each site is a fraction of the total 300-425 micrograms. For example, one may implant a single does of 324 micrograms at one site, or two separate doses of 162 micrograms at two sites, or three separate dose of 108 micrograms at three sites that triangulate the tissue site. It is important to limit the total dosage to an amount less than that which would be harmful to the organism. However, in some embodiments, although when there are a plurality of sites each site may contain less than the total dose that might have been administered in a single application, it is important to remember that each site will independent have a release profile, and the biopolymers' concentration and substance should be adjusted accordingly to ensure that the sustain release occurs over sufficient time.

In some embodiments, there is a drug depot comprising clonidine or clonidine hydrochloride and a polymer, wherein the polymer is one more of various embodiments, the drug depot comprises poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone or a combination thereof.

In one exemplary dosing regimen, a rat may be provided with sufficient clonidine in a biodegradable polymer to provide sustain release of 0.240 μg/day for 135 days. The total amount of clonidine that is administered over this time period would be approximately 32.4 μg. In another exemplary dosing regimen, a human is provided with sufficient clonidine in a biodegradable polymer to provide sustain release of 2.4 μg/day for 135 days. The total amount of clonidine that is administered over this time period would be approximately 324 μg.

When using a plurality of pellets, the pellet number is based on the amount of drug loading into a pellet of appropriate size (i.e., 0.5 mm diameter×4 mm length) and how much drug is needed (e.g., approximately 325 μg clonidine (3 pellets)). In some embodiments there is a polymer that releases a bolus amount of compound over the first few (˜5) days before it settles down and releases 2.5 mg/day for 135 days. An exemplary formulation is 5% wt. clonidine, 100 DL 5E.

In some embodiments, the polymer depots of present application enable one to provide efficacy of the active ingredient that is equivalent to subcutaneous injections that deliver more than 2.5 times as much drug.

### Bupivacaine

The drug depot may comprise the analgesic bupivacaine. When referring to bupivacaine, unless otherwise specified or apparent from context it is understood that the inventor is also referring to pharmaceutically acceptable salts. Some examples of potentially pharmaceutically acceptable salts include those salt-forming acids and bases that do not substantially increase the toxicity of the compound. Some examples of these salts include salts of alkali metals such as magnesium, potassium and ammonium. Salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, and the like. To the extent these salts of bupivacaine can be created for safe administration to a mammal, they are within the scope of the present invention.

Further, the bupivacaine may also be used in a base form. In various embodiments, the drug depot releases about 1 mg to 30 mg/day of bupivacaine for 1 to 10 days or 1 day to 6 months. In some embodiments it releases 20 to 360 mg/day or 40 to 120 mg/day or 80 to 180 mg/day or 120 to 240 mg/day or 160 to 300 mg/day or 200 to 360 mg/day or bupivacaine. This dose is often much lower than the dose used to provide nerve block in surgery.

In some embodiments, the amount of bupivacaine is between 2 mg/day to 1800 mg/day or between 10 and 1500 mg/day. The release of the bupivacaine may be for at least three, at least four at least five, at least six, at least seven or at least eight days in the recited ranges.

### Fluocinolone

In one embodiment, the anti-inflammatory agent in the drug depot comprises fluocinolone or a pharmaceutically acceptable salt thereof such as the acetonide salt. Fluocinolone is available from various pharmaceutical manufacturers. The dosage of fluocinolone may be from approximately 0.0005 to approximately 100 μg/day. Additional dosages of fluocinolone include from approximately 0.0005 to approximately 50 μg/day; approximately 0.0005 to approximately 25 μg/day; approximately 0.0005 to approximately 10 μg/day; approximately 0.0005 to approximately 5 μg/day; approximately 0.0005 to approximately 1 μg/day; approximately 0.0005 to approximately 0.75 μg/day; approximately 0.0005 to approximately 0.5 μg/day; approximately 0.0005 to approximately 0.25 μg/day; approximately 0.0005 to approximately 0.1 μg/day; approximately 0.0005 to approximately 0.075 μg/day; approximately 0.0005 to approximately 0.05 μg/day; approximately 0.001 to approximately 0.025 μg/day; approximately 0.001 to approximately 0.01 μg/day; approximately 0.001 to approximately 0.0075 μg/day; approximately 0.001 to approximately 0.005 μg/day; approximately 0.001 to approximately 0.025 μg/day; and approximately 0.002 μg/day. In another embodiment, the dosage of fluocinolone is from approximately 0.001 to approximately 15 μg/day. In another embodiment, the dosage of fluocinolone is from approximately 0.001 to approximately 10 μg/day. In another embodiment, the dosage of fluocinolone is from approximately 0.001 to approximately 5 μg/day. In another embodiment, the dosage of fluocinolone is from approximately 0.001 to 2.5 μg/day. In some embodiments, the amount of fluocinolone is between 40 and 600 μg/day. In some embodiments, the amount of fluocinolone is between 200 and 400 μg/day.

### Dexamethasone

In one embodiment, the anti-inflammatory agent in the drug depot is dexamethasone free base or dexamethasone acetate, also referred to as 8S,9R,10S,11S,13S,14S,16R,17R)-9-Fluoro-11,17-dihydroxy-17-(2-hydroxyacetyl)-10,13,16-trimethyl-6,7,8,11,12,14,15,16 octahydrocyclopenta[a]-phenanthren-3-one), or a pharmaceutically acceptable salt thereof, which is available from various manufacturers.

In various embodiments, dexamethasone may be released from the depot at a dose of about 10 pg to about 80 mg/day, about 2.4 ng/day to about 50 mg/day, about 50 ng/day to about 2.5 mg/day, about 250 ng/day to about 250 ug/day, about 250 ng/day to about 50 ug/day, about 250 ng/day to about 25 ug/day, about 250 ng/day to about 1 ug/day, about 300 ng/day to about 750 ng/day or about 0.50 ug/day. In various embodiments, the dose may be about 0.01 to about 10 μg/day or about 1 ng to about 120 μg/day.

In one exemplary embodiment, the dexamethasone is dexamethasone sodium phosphate.

### GED

In one embodiment, the anti-inflammatory agent in the drug depot is GED (guanidinoethyldisulfide), which is an inducible nitric oxide synthase inhibitor having anti-inflammatory properties. GED may be in its hydrogen carbonate salt form.

The dosage of GED may be from approximately 0.0005 μg/day to approximately 100 mg/day. Additional dosages of GED include from approximately 0.0005 μg/day to approximately 50 mg/day; approximately 0.0005 μg/day to approximately 10 mg/day; approximately 0.0005 μg/day to approximately 1 mg/day; approximately 0.0005 to approximately 800 μg/day; approximately 0.0005 to approximately 50 μg/day; approximately 0.001 to approximately 45 μg/day; approximately 0.001 to approximately 40 μg/day; approximately 0.001 to approximately 35 μg/day; approximately 0.0025 to approximately 30 μg/day; approximately 0.0025 to approximately 25 μg/day; approximately 0.0025 to approximately 20 μg/day; and approximately 0.0025 to approximately 15 μg/day. In another embodiment, the dosage of GED is from approximately 0.005 to approximately 15 μg/day. In another embodiment, the dosage of GED is from approximately 0.005 to approximately 10 μg/day. In another embodiment, the dosage of GED is from approximately 0.005 to approximately 5 μg/day. In another embodiment, the dosage of GED is from approximately 0.005 to 2.5 μg/day. In some embodiments, the amount of GED is between 40 and 600 μg/day. In some embodiments, the amount of GED is between 200 and 400 μg/day.

In one exemplary embodiment the dosage of GED is between 0.5 and 4 mg/day. In another exemplary embodiment the dosage of GED is between 0.75 and 3.5 mg/day.

### Lovastatin

In one exemplary embodiment, the anti-inflammatory agent in the drug depot comprises lovastatin. Lovastatin is a statin that may be obtained from various manufacturers in various forms (e.g., injection, powder, etc.). For example, lovastatin may be obtained from Merck as Mevacor® (see U.S. Pat. No. 4,231,938, the entire disclosure is herein incorporated by reference). Suitable pharmaceutically acceptable salts of lovastatin include one or more compounds derived from bases such as sodium hydroxide, potassium hydroxide, lithium hydroxide, calcium hydroxide, 1-deoxy-2-(methylamino)-D-glucitol, magnesium hydroxide, zinc hydroxide, aluminum hydroxide, ferrous or ferric hydroxide, ammonium hydroxide or organic amines such as N-methylglucamine, choline, arginine or the like or combinations thereof. Suitable pharmaceutically acceptable salts of lovastatin include lithium, calcium, hemicalcium, sodium, potassium, magnesium, aluminum, ferrous or ferric salts thereof or a combination thereof.

In various embodiments, the therapeutically effective amount of lovastatin comprises from about 0.1 pg to about 2000 mg, for example, 0.1 ng to 1000 mg, 500 mg, 100 mg, 50 mg, 25 mg, 10 mg, 1 mg, 50 μg, 25 μg, 10 μg, 1 μg, 500 ng, 250 ng, 100 ng, 75 ng, 50 ng, 25 ng, 15 ng, 10 ng, 5 ng, or 1 ng of lovastatin per day. In various embodiments, the dosage may be, for example from about 3 ng/day to 0.3 μg/day.

### Morphine

In one embodiment of the present invention, the analgesic agent in the drug depot is morphine. Morphine is also referred to as (5α,6α)-7,8-didehydro-4,5-epoxy-17-methylmorphinan-3,6-diol and has the chemical formula C₁₇H₁₉NO₃. Morphine or a pharmaceutically acceptable salt thereof is available from various manufacturers. In one exemplary embodiment, the morphine comprises morphine sulfate or hydrochloride.

The dosage of the morphine may be from 0.1 mg to 1000 mg per day. For example, the dosage of morphine may be for example, 0.1 mg to 2 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 75 mg, 80 mg, 85 mg, 90 mg, 95 mg, 100 mg, 120 mg, 130 mg, 140 mg, 150 mg, 160 mg, 170 mg, 180 mg, 190 mg, 200 mg of morphine per day.

### Tramadol

In one embodiment, the analgesic agent in the drug depot is tramadol. Tramadol is also referred to as (±)cis-2-[(dimethylamino)methyl]-1-(3-methoxyphenyl) cyclohexanol hydrochloride and has the chemical formula C₁₆H₂₅NO₂. Tramadol or a pharmaceutically acceptable salt thereof is available from various manufacturers. In various embodiments, tramadol HCL was used.

The dosage of the tramadol may be from 0.01 mg to 500 mg per day. For example, the dosage of tramadol may be for example, 0.1 mg to 2 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 75 mg, 80 mg, 85 mg, 90 mg, 95 mg, 100 mg, 120 mg, 130 mg, 140 mg, 150 mg, 160 mg, 170 mg, 180 mg, 190 mg, 200 mg, or 500 mg of tramadol per day.

In one embodiment, the drug depot contains sufficient tramadol to release between 2.5 and 30 mg/kg/day. In another embodiment the drug depot contains sufficient tramadol to release between 3 and 27.5 mg/kg/day.

### Reversible Phase Transition Material

The drug depot comprises an analgesic and/or anti-inflammatory disposed in a reversible phase transition material, which changes phases or physical state (e.g., solid to liquid, solid to semi-solid, semi-solid to liquid, liquid to solid, liquid to semi-solid, or semi-solid to solid, glass to rubber, crystal to melt, semi-crystal to melt, etc.) in response to an external stimuli, such as for example, change in temperature. For example, the drug depot may comprise entirely or in one or more layer(s) a reversible phase transition material having an analgesic and/or anti-inflammatory agent as discussed above disposed in the reversible phase transition material. When heat is applied to the drug depot (e.g., 40° C. to 45° C.) or the skin next to where the drug depot is implanted, this will cause the reversible phase transition material in an implanted drug depot to change, for example, from solid to liquid or solid to semi-solid or semi-solid to liquid and thus increase drug diffusion across the depot and cause release of a bolus dose, or burst dose of the analgesic and/or anti-inflammatory agent from the drug depot. In this way, breakthrough pain and/or inflammation can be reduced, prevented or treated. By disposing the reversible phase transition material in the entire drug depot or in one or more layer(s) of the drug depot, a burst effect can be accomplished where the drug depot will release a bolus dose in 5, 10, 15, 20, 25, 30, 25, 40, 45, 50, 55, 60, 75, 90, 100, 120, 140, 160, or 180 minutes during or after the heat is applied to prevent, reduce and/or treat pain and/or inflammation at a target tissue site. In some embodiments, the application of heat to the drug depot will increase the solubility of the drug in the polymer and this may also increase release.

As another example, when cold is applied to the drug depot (e.g., 20° C. to 25° C.) or the skin next to where the drug depot is implanted (which may be 0.5 mm to 5 cm away from the drug depot), this will cause the reversible phase transition material in an implanted drug depot to change from a water insoluble or solid phase to a water soluble phase or liquid where the drug can be released when the temperature is lowered. In some embodiments, the colder temperature can cause the reversible phase transition polymer to reach its glass transition temperature but this would slow release.

In some embodiments, the phase changes can be solid to liquid or solid to semi-solid or semi-solid to liquid or liquid to semi solid, or liquid to solid, or semi-solid to solid and thus increase drug diffusion causing increase release of a bolus dose, or burst dose of the analgesic and/or anti-inflammatory agent from the drug depot. In this way, breakthrough pain and/or inflammation can be reduced, prevented or treated. By disposing the reversible phase transition material in the entire drug depot or in one or more layer(s) of the drug depot, a burst effect can be accomplished where the drug depot will release a bolus dose in 5, 10, 15, 20, 25, 30, 25, 40, 45, 50, 55, 60, 75, 90, 100, 120, 140, 160, or 180 minutes during or after the cold is applied to prevent, reduce and/or treat pain and/or inflammation at a target tissue site.

The reversible phase transition material can include biodegradable polymeric and non-polymeric material. Examples of material suitable for use as the reversible phase transition material include paraffin waxes, poloxamers, polylactones, paraffin waxes, poly(N-isopropylacrylamide) homopolymer, poly(N-isopropylacrylamide)acrylamide copolymer, copolymer of poly(N-isopropylacrylamide) containing silane monomers selected from [3-(methacryloyloxy)propyl]trimethoxysilane, [2-(methacryloyloxy)ethoxy]-trimethylsilane and methacryloyloxy)trimethylsilane, copolymer of poly(hydroxypropyl methacrylamide), dicarboxymethylaminopropyl methacrylamide, xyloglucan, ethyl(hydroxyethyl)cellulose, poly(ethyleneoxide-b-propylene oxide-b-ethylene oxide) and its copolymers, poly(ethylene oxide)/(D,L-lactic acid-co-glycolic acid)copolymers, combinations of chitosan and polyol salts, poly(silamine), and poly(organophosphazene) poly(alpha-hydroxy acids), poly(lactide-co-glycolide) (PLGA or PLG), polylactide (PLA), polyglycolide (PG), polyethylene glycol (PEG), mPEG, PEG conjugates of poly(alpha-hydroxy acids), polyorthoesters, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide-ε-caprolactone, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) or combinations thereof. Suitable reversible phase transition material for use in the drug depot is described in U.S. Pat. No. 5,226,902. The entire disclosure of this patent is hereby incorporated by reference into the present application.

In some embodiments, the reversible phase transition material comprises a thermosensitive hydrogel that swells or shrink in response to changes in temperature. For example, in certain embodiments, the analgesic and/or anti-inflammatory agent incorporated in such a hydrogel will be released when the hydrogel shrinks in response to temperature change, e.g. by heating. Conversely, when such a hydrogel is subsequently cooled to an appropriate temperature at which it re-swells, residual drug in the drug depot will be re-incorporated back into the hydrogel and thus release will be decreased. Also, as cold is applied, in this embodiment, the drug will be less soluble in the polymer and there will be less release from the drug depot. Accordingly, the availability and/or release of the drug from the hydrogel can be controlled.

Hydrogels include natural hydrogels, such as, for example, gelatin, collagen, silk, elastin, fibrin and polysaccharide-derived polymers like agarose, and chitosan, glucomannan gel, hyaluronic acid, polysaccharides, such as cross-linked carboxyl-containing polysaccharides, or a combination thereof. Synthetic hydrogels include, but are not limited to those formed from polyvinyl alcohol, acrylamides such as polyacrylic acid and poly (acrylonitrile-acrylic acid), polyurethanes, polyethylene glycol (e.g., PEG 3350, PEG 4500, PEG 8000), silicone, polyolefins such as polyisobutylene and polyisoprene, copolymers of silicone and polyurethane, neoprene, nitrile, vulcanized rubber, poly(N-vinyl-2-pyrrolidone), acrylates such as poly(2-hydroxy ethyl methacrylate) and copolymers of acrylates with N-vinyl pyrolidone, N-vinyl lactams, polyacrylonitrile or combinations thereof. The hydrogel materials may further be cross-linked to provide further strength as needed. Examples of different types of polyurethanes include thermoplastic or thermoset polyurethanes, aliphatic or aromatic polyurethanes, polyetherurethane, polycarbonate-urethane or silicone polyether-urethane, or a combination thereof.

In various embodiments, the polymers (including the reversible phase transition material and/or the sustain release polymer) may comprise at least 98 or 99.5 wt %, at least 95 wt %, at least 90 wt %, at least 85 wt %, at least 80 wt %, at least 75 wt %, at least 70 wt %, at least 65 wt %, at least 60 wt %, at least 55 wt %, at least 50 wt %, at least 45 wt %, at least 40 wt %, at least 35 wt %, at least 30 wt %, at least 25 wt %, at least 20 wt %, at least 15 wt %, at least 10 wt %, at least 5 wt % of the drug depot.

In some embodiments, for the reversible phase transition material, the glass transition temperature (Tg) for the material may be one parameter needed for the targeted controlled delivery of the analgesic and/or anti-inflammatory agent. When the drug depot temperature is above the glass transition temperature Tg, in some embodiments, the polymer becomes rubbery thus increasing the drug diffusion coefficient and the permeability of the reversible phase transition material, which increases drug release from the reversible phase transition material and thus drug release from the drug depot. In some embodiments, when the drug depot temperature is below the Tg, the reversible phase transition polymer becomes glassy this will cause decreases in the drug diffusion coefficient and permeability and thus decrease release from the reversible phase transition material and the drug depot. In some embodiments, the reversible phase transition material and/or the drug depot has a Tg that is lower than body temperature (36° C.-38° C.). In some embodiments, the reversible phase transition material and/or the drug depot has a Tg that is higher than body temperature (36° C.-38° C.).

In some embodiments, the Tg is between about 30° C.-40° C. so that the transition can be achieved at a temperature that does not burn the patient. A Tg that is too low may for the polymer will mean that the polymer is very rubbery and heating the area will have a limited effect on increasing the release rate.

The drug depot can comprise the reversible phase transition material in combination with one or more biodegradable polymers that provide the desired properties for reversible phase transition and the desired sustain release properties for the analgesic and/or anti-inflammatory agent. For example, in some embodiments, the drug depot may have a reversible phase transition material in one or more layer(s) that may release a bolus dose of the analgesic and/or anti-inflammatory agent at a site beneath the skin when heat, cold or another suitable form of energy, e.g., ultrasound energy, light, mechanical energy (such as agitation or massage), electrical, chemical, or magnetic energy is applied to it. This will be the immediate release layer(s) and drug depot may have one or more sustain release layer(s), as well, which are biodegradable and may release the analgesic and/or anti-inflammatory agent over a longer period of up to 10 days. In this way post-operative pain and/or inflammation and breakthrough pain and/or inflammation can be treated simultaneously.

It has also been found that the use of produces a positive, synergistic effect on the targeted, controlled delivery of the analgesic and/or anti-inflammatory agent from the drug depot. Not only does the patient feel the soothing on the skin, but the will increase release from the drug depot and provide an “extra dose”, which will provide additional relief.

The cold can be applied to the skin near the site the drug depot has been implanted by any cold source. The cold source will transfer cold through the skin and to the area around the drug depot and to the drug depot itself to cause change in the reversible phase transition material (e.g., solid to liquid, solid to semi-solid, semi-solid to liquid, liquid to solid, liquid to semi-solid, or semi-solid to solid) to cause increase or decrease release of the analgesic and/or anti-inflammatory from the drug depot. Suitable cold sources include ice packs, cold packs, cold liquid, or endothermic cold packs, endothermic cold pads, electric cold pads or electric cold packs, or the like. The cold (e.g., 0° C. to 30° C.) brings the temperature of skin lower than body temperature and when the drug depot reaches a temperature of 0° C. to 30° C., this causes an increase or decrease release of the drug from the drug depot.

The heat can be applied to the skin near the site the drug depot has been implanted by any heat source. The heat source will transfer heat through the skin and to environment surrounding the drug depot and then to the drug depot itself to cause change in the reversible phase transition material (e.g., solid to liquid, solid to semi-solid, semi-solid to liquid, liquid to solid, liquid to semi-solid, or semi-solid to solid) which causes increase or decrease release of the analgesic and/or anti-inflammatory from the drug depot. Suitable heat sources include heat packs, heating pads, hot liquid, or exothermic heat packs, exothermic head pads, electric heating pads, electric heating packs, or the like. The heat (e.g., greater than 39° C.) brings the temperature of skin higher than body temperature and when the drug depot reaches a temperature of greater than 39° C. (e.g., 40° C. to 45° C.), this causes an increase or decrease release of the drug from the drug depot.

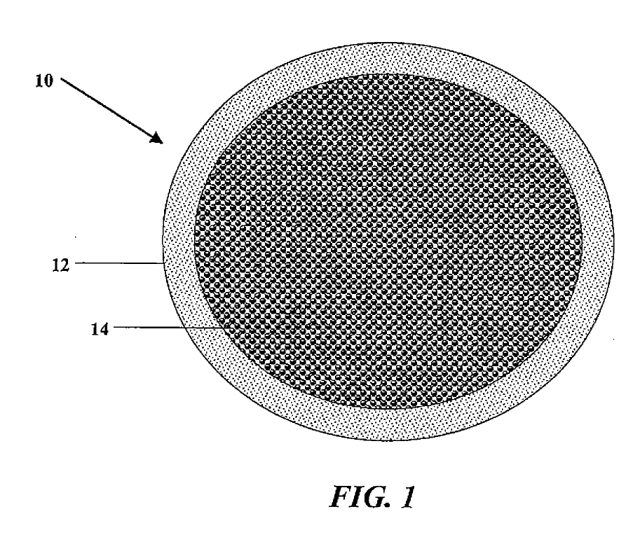

FIG. 1 is a magnified side sectional view of an embodiment of the implantable drug depot 10 having a layer of the reversible phase transition material 12 holding the drug 14 (e.g., anti-inflammatory, muscle relaxant, and/or analgesic) within the drug depot. In this illustrated embodiment, the drug depot has not had heat, cold or another suitable form of energy, e.g., ultrasound energy, applied to it and the drug will be exhibit sustained release characteristics over time.

As heat, cold or another suitable form of energy, e.g., ultrasound energy, is applied to the skin of the patient near the site where the drug depot has been implanted. The heat, cold or another suitable form of energy, e.g., ultrasound energy, will travel through the skin and to environment surrounding the implanted drug depot and then to the drug depot itself. This will cause release of a bolus dose or a burst release of the drug.

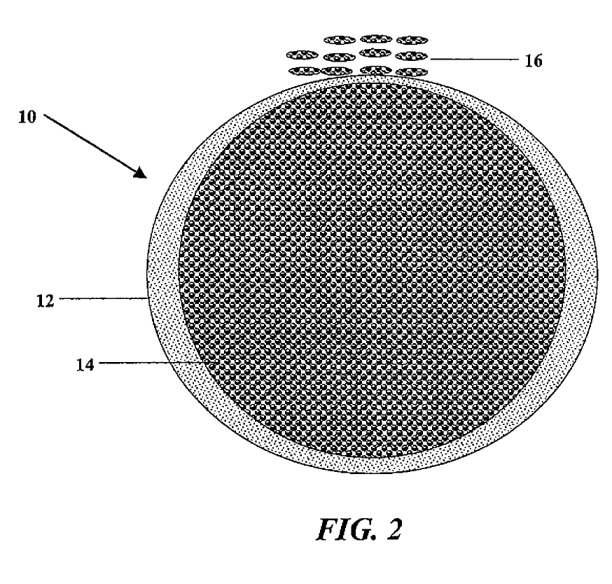

FIG. 2 is a magnified side sectional view of an embodiment of the implantable drug depot 10 having a layer of the reversible phase transition material 12 that is changing to a liquid state causing release of the analgesic and/or anti-inflammatory agent 14 from the drug depot as heat is applied to it. The heat will travel through the skin of the patient and to environment surrounding the implanted drug depot and then to the drug depot itself. This will cause release of a bolus dose or a burst release of the drug 16 at the target tissue site providing the patient with an “extra dose” of the analgesic and/or anti-inflammatory agent, which will cause added relief. Such embodiment is particularly useful to reduce, treat or prevent post-operative breakthrough pain. For example, following surgery, “breakthrough pain” (a suddenly increased and relatively short lasting pain, in addition to a continuous “baseline” pain) may occur. With the help of the temperature controlled drug depot, breakthrough pain can be controlled. When an episode of breakthrough pain occurs, to deliver more of the analgesic and/or anti-inflammatory agent to the target tissue site, the patient or health practitioner applies a heat source (e.g., heat patch) or cold source (e.g., cold patch) to the skin of the patient or the site where the drug depot has been implanted until the pain and/or inflammation is alleviated. The duration of the heating patch or cold patch is preferably designed to be long enough to deliver sufficient extra analgesic and/or anti-inflammatory agent, but not long enough to deliver the extra amount of the analgesic and/or anti-inflammatory agent that may pose a risk to the patient. The patient and/or health practitioner may also remove the heat patch or cold patch when the breakthrough pain begins to diminish.

The present application is suitable for use in patient controlled analgesia (hereinafter “PCA”), in which the patient gives himself or herself a dose of analgesic and/or anti-inflammatory when he/she feels the need. The ranges of the dose and dosing frequency are usually set by a health practitioner (i.e., caring physician, nurse, etc.). In many PCA situations, the patient receives a baseline rate of analgesic and/or anti-inflammatory, and gets extra bolus analgesic and/or analgesic when he/she feels that it is needed. The technology in the present application may be used for a PCA in which the patient gets the baseline dose from the drug depot and the extra (“rescue”) dose or bolus dose by heating or cooling the skin area where the drug depot was implanted. For example, the drug depot may be implanted within 1 to 5 mm of an epidermis, dermis, or subcutaneous tissue and heat, cold or another suitable form of energy, e.g., ultrasound energy, is applied to the skin to cause release of the bolus dose of the analgesic, muscle relaxant and/or the anti-inflammatory agent from the drug depot into this area. In this way, the drug depot provides immediate release and sustained release treatment of pain and/or inflammation.

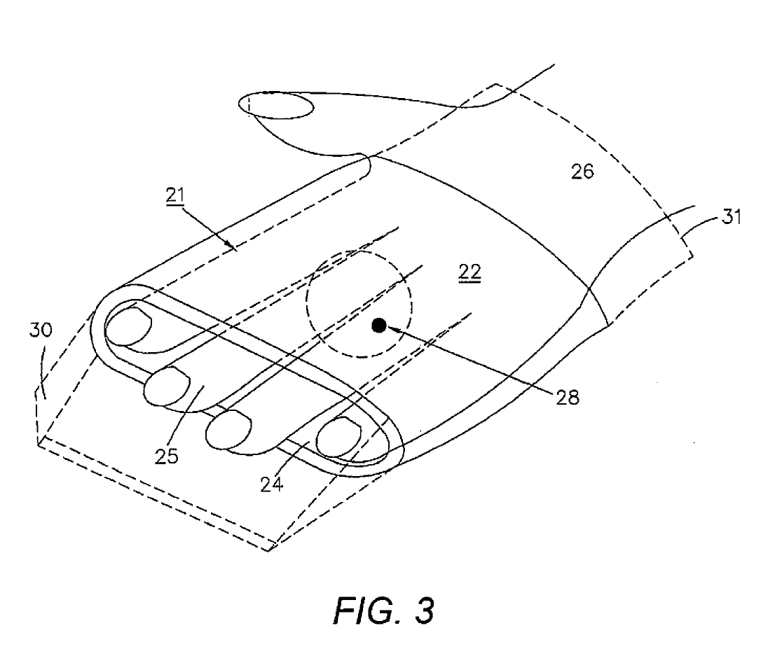

FIG. 3 is a perspective view of one embodiment illustrating a cold or hot pack 31 being applied after hand surgery to the hand 26 near the area that the drug depot 28 was implanted underneath the skin 22. The cold or hot pack 31 has an opening 24 to insert the hand 26, where fingers 21 and 25 can be inserted into the opening. The cold or hot pack can have optionally a stand 30 to immobilize the hand while it is in the pack. Here the application of cold or heat to the skin area 22 causes the heat, cold or another suitable form of energy, e.g., ultrasound energy, to be transmitted to the environment surrounding the drug depot and the drug depot 28, which causes the reversible phase transition material to change phases (e.g., solid to liquid, solid to semi-solid, semi-solid to liquid, etc.) to release a bolus dose or “rescue dose” or “extra dose” of the analgesic and/or anti-inflammatory agent to the patient, which will reduce, prevent or treat an episode of pain and/or inflammation. The patient may also remove the cold or heat pack when the pain and/or inflammation begins to diminish and the drug depot will go back to releasing the analgesic and/or anti-inflammatory agent over the scheduled sustain release duration. Although the hand area is shown, the drug depot can be delivered to any site beneath the skin, including, but not limited to, at least one muscle, ligament, tendon, cartilage, foot, finger, toe, hand, wrist, gum, jaw, knee joint, spinal disc, spinal foraminal space, near the spinal nerve root, or spinal canal.

Typically, the depot will be a solid or semi-solid formulation comprising a biocompatible material that can be biodegradable. The term “solid” is intended to mean a rigid material, while “semi-solid” is intended to mean a material that has some degree of flexibility, thereby allowing the depot to bend and conform to the surrounding tissue requirements. An example of a semi-solid material is a gel. The term liquid includes, solutions, suspensions and/or slurries containing the therapeutic agent.

In various embodiments, the drug depot may not be biodegradable. For example, the drug depot may comprise polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. Typically, these types of drug depots may need to be removed.

### Biodegradable Depots

In some instance, it may be desirable to avoid having to remove the drug depot after use. In those instances, the depot may comprise a biodegradable material. There are numerous materials available for this purpose and having the characteristic of being able to breakdown or disintegrate over a prolonged period of time when positioned at or near the target tissue. As a function of the chemistry of the biodegradable material, the mechanism of the degradation process can be hydrolytical or enzymatical in nature, or both. In various embodiments, the degradation can occur either at the surface (heterogeneous or surface erosion) or uniformly throughout the drug delivery system depot (homogeneous or bulk erosion).

In various embodiments, the depot may comprise a bioabsorbable, and/or a biodegradable biopolymer that may provide immediate release, or sustained release of the at least one analgesic agent and/or at least one anti-inflammatory agent. Examples of suitable sustained release biopolymers include but are not limited to poly(alpha-hydroxy acids), poly (lactide-co-glycolide) (PLGA or PLG), polylactide (PLA), polyglycolide (PG), polyethylene glycol (PEG) conjugates of poly(alpha-hydroxy acids), polyorthoesters, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, ,-caprolactone, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) or combinations thereof. As persons of ordinary skill are aware, mPEG may be used as a plasticizer for PLGA, but other polymers/excipients may be used to achieve the same effect. mPEG imparts malleability to the resulting formulations.

Where different combinations of polymers are used (bi, tri (e.g., PLGA-PEO-PLGA) or terpolymers), they may be used in different molar ratios, 1:1, 2:1, 3:1, 4:1, 5:1, 6:1, 7:1, 8:1, 9:1, or 10:1. In various embodiments, for the 130 day release, the depot comprises 50:50 PLGA to 100 PLA. The molecular weight range is 0.45 to 0.8 dI/g.

In various embodiments, the molecular weight of the polymer can be a wide range of values. The average molecular weight of the polymer can be from about 1000 to about 10,000,000; or about 1,000 to about 1,000,000; or about 5,000 to about 500,000; or about 10,000 to about 100,000; or about 20,000 to 50,000.

In some embodiments, the at least one biodegradable polymer comprises poly(lactic-co-glycolic acid) (PLA) or poly(orthoester) (POE) or a combination thereof. The poly(lactic-co-glycolic acid) may comprise a mixture of polyglycolide (PGA) and polylactide and in some embodiments, in the mixture, there is more polylactide than polyglycolide. In various other embodiments there is 100% polylactide and 0% polyglycolide; 95% polylactide and 5% polyglycolide; 90% polylactide and 10% polyglycolide; 85% polylactide and 15% polyglycolide; 80% polylactide and 20% polyglycolide; 75% polylactide and 25% polyglycolide; 70% polylactide and 30% polyglycolide; 65% polylactide and 35% polyglycolide; 60% polylactide and 40% polyglycolide; 55% polylactide and 45% polyglycolide; 50% polylactide and 50% polyglycolide; 45% polylactide and 55% polyglycolide; 40% polylactide and 60% polyglycolide; 35% polylactide and 65% polyglycolide; 30% polylactide and 70% polyglycolide; 25% polylactide and 75% polyglycolide; 20% polylactide and 80% polyglycolide; 15% polylactide and 85% polyglycolide; 10% polylactide and 90% polyglycolide; 5% polylactide and 95% polyglycolide; and 0% polylactide and 100% polyglycolide.

In various embodiments that comprise both polylactide and polyglycolide; there is at least 95% polylactide; at least 90% polylactide; at least 85% polylactide; at least 80% polylactide; at least 75% polylactide; at least 70% polylactide; at least 65% polylactide; at least 60% polylactide; at least 55%; at least 50% polylactide; at least 45% polylactide; at least 40% polylactide; at least 35% polylactide; at least 30% polylactide; at least 25% polylactide; at least 20% polylactide; at least 15% polylactide; at least 10% polylactide; or at least 5% polylactide; and the remainder of the biopolymer being polyglycolide.

In various embodiments, the drug depot comprises poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone, glycolide-caprolactone or a combination thereof.

As persons of ordinary skill in the art are aware, implantable elastomeric depot compositions having a blend of polymers with different end groups are used the resulting formulation will have a lower burst index and a regulated duration of delivery. For example, one may use polymers with acid (e.g., carboxylic acid) and ester end groups (e.g., lauryl, methyl or ethyl ester end groups).

Additionally, by varying the comonomer ratio of the various monomers that form a polymer (e.g., the L/G/CL or G/CL ratio for a given polymer) there will be a resulting depot composition having a regulated burst index and duration of delivery. For example, a depot composition having a polymer with a L/G ratio of 50:50 may have a short duration of delivery ranging from about two days to about one month; a depot composition having a polymer with a L/G ratio of 65:35 may have a duration of delivery of about two months; a depot composition having a polymer with a L/G ratio of 75:25 or L/CL ratio of 75:25 may have a duration of delivery of about three months to about four months; a depot composition having a polymer ratio with a L/G ratio of 85:15 may have a duration of delivery of about five months; a depot composition having a polymer with a L/CL ratio of 25:75 or PLA may have a duration of delivery greater than or equal to six months; a depot composition having a terpolymer of CL/G/L (CL refers to caprolactone, G refers to glycolic acid and L refers to lactic acid) with G greater than 50% and L greater than 10% may have a duration of delivery of about one month and a depot composition having a terpolymer of CL/G/L with G less than 50% and L less than 10% may have a duration months up to six months. In general, increasing the G content relative to the CL content shortens the duration of delivery whereas increasing the CL content relative to the G content lengthens the duration of delivery.

In some embodiments, the biodegradable polymer comprises at least 10 wt %, at least 50 wt. %, at least 60 wt. %, at least 70 wt. %, at least 80 wt. %, at least 85 wt. %, at least 90 wt. %, at least 95 wt. %, or at least 99 wt. % of the formulation. In some embodiments, the at least one biodegradable polymer and the at least one analgesic and/or anti-inflammatory agent are the only components of the pharmaceutical formulation.

In some embodiments, at least 75% of the particles in the drug depot have a size from about 1 micrometer to about 200 micrometers. In some embodiments, at least 85% of the particles in the drug depot have a size from about 1 micrometer to about 100 micrometers. In some embodiments, at least 95% of the particles in the drug depot have a size from about 5 micrometer to about 50 micrometers. In some embodiments, all of the particles in the drug depot have a size from about 10 micrometer to about 50 micrometers.

In some embodiments, at least 75% of the particles in the drug depot have a size from about 5 micrometer to about 20 micrometers. In some embodiments, at least 85% of the particles in the drug depot have a size from about 5 micrometers to about 20 micrometers. In some embodiments, at least 95% of the particles in the drug depot have a size from about 5 micrometer to about 20 micrometers. In some embodiments, all of the particles in the drug depot have a size from about 5 micrometer to about 20 micrometers.

The depot may optionally contain inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfite, sodium bisulfate, sodium thiosulfate, thimerosal, methylparaben, polyvinyl alcohol and phenylethyl alcohol; solubility adjusting agents; stabilizers; and/or cohesion modifiers. These inactive ingredients may have multi-functional purposes including the carrying, stabilizing and controlling the release of the therapeutic agent(s). The sustained release process, for example, may be by a solution-diffusion mechanism or it may be governed by an erosion-sustained process. Typically, any such inactive materials will be present within the range of 0-75 wt %, and more typically within the range of 0-30 wt %. If the depot is to be placed in the spinal area, in various embodiments, the depot may comprise sterile preservative free material.

The depot can be different sizes, shapes and configurations. There are several factors that can be taken into consideration in determining the size, shape and configuration of the drug depot. For example, both the size and shape may allow for ease in positioning the drug depot at the target tissue site that is selected as the implantation or injection site. In addition, the shape and size of the system should be selected so as to minimize or prevent the drug depot from moving after implantation or injection. In various embodiments, the drug depot can be shaped like a pellet, a sphere, a cylinder such as a rod or fiber, a flat surface such as a disc, film or sheet or the like. Flexibility may be a consideration so as to facilitate placement of the drug depot. In various embodiments, the drug depot can be different sizes, for example, the drug depot may be a length of from about 0.5 mm to 5 mm and have a diameter of from about 0.01 to about 2 mm. In various embodiments, the drug depot may have a layer thickness of from about 0.005 to 1.0 mm, such as, for example, from 0.05 to 0.75 mm.

In various embodiments, when the drug depot comprises a pellet, it may be placed at the incision site before the site is closed. The pellet may for example be made of thermoplastic materials. Additionally, specific materials that may be advantageous for use in the pellet include but are not limited to the compounds identified above as sustained release biopolymers. The drug depot may be formed by mixing the at least one analgesic and/or anti-inflammatory agent with the polymer.

Radiographic markers can be included on the drug depot to permit the user to position the depot accurately into the target site of the patient. These radiographic markers will also permit the user to track movement and degradation of the depot at the site over time. In this embodiment, the user may accurately position the depot in the site using any of the numerous diagnostic imaging procedures. Such diagnostic imaging procedures include, for example, X-ray imaging or fluoroscopy. Examples of such radiographic markers include, but are not limited to, barium, calcium phosphate, and/or metal beads or particles. In various embodiments, the radiographic marker could be a spherical shape or a ring around the depot.

### Gel

In various embodiments, the drug depot comprises a gel having a pre-dosed viscosity in the range of about 1 to about 500 centipoise (cps), 1 to about 200 cps, or 1 to about 100 cps. After the gel is administered to the target site, the viscosity of the gel will increase and the gel will have a modulus of elasticity (Young's modulus) in the range of about 1×10⁴ to about 6×10⁵ dynes/cm², or 2×10⁴ to about 5×10⁵ dynes/cm², or 5×10⁴ to about 5×10⁵ dynes/cm².

In one embodiment, a depot is provided that contains an adherent gel comprising at least one analgesic and/or anti-inflammatory agent that is evenly distributed throughout the gel. The gel may be of any suitable type, as previously indicated, and should be sufficiently viscous so as to prevent the gel from migrating from the targeted delivery site once deployed; the gel should, in effect, “stick” or adhere to the targeted tissue site. The gel may, for example, solidify upon contact with the targeted tissue or after deployment from a targeted delivery system. The targeted delivery system may be, for example, a syringe, a catheter, needle or cannula or any other suitable device. The targeted delivery system may inject the gel into or on the targeted tissue site. The therapeutic agent may be mixed into the gel prior to the gel being deployed at the targeted tissue site. In various embodiments, the gel may be part of a two-component delivery system and when the two components are mixed, a chemical process is activated to form the gel and cause it to stick or to adhere to the target tissue.

In various embodiments, a gel is provided that hardens or stiffens after delivery. Typically, hardening gel formulations may have a pre-dosed modulus of elasticity in the range of about 1×10⁴ to about 3×10⁵ dynes/cm², or 2×10⁴ to about 2×10⁵ dynes/cm², or 5×10⁴ to about 1×10⁵ dynes/cm². The post-dosed hardening gels (after delivery) may have a rubbery consistency and have a modulus of elasticity in the range of about 1×10⁴ to about 2×10⁶ dynes/cm², or 1×10⁵ to about 7×10⁵ dynes/cm², or 2×10⁵ to about 5×10⁵ dynes/cm².

In various embodiments, for those gel formulations that contain a polymer, the polymer concentration may affect the rate at which the gel hardens (e.g., a gel with a higher concentration of polymer may coagulate more quickly than gels having a lower concentration of polymer). In various embodiments, when the gel hardens, the resulting matrix is solid but is also able to conform to the irregular surface of the tissue (e.g., recesses and/or projections in bone).

The percentage of polymer present in the gel may also affect the viscosity of the polymeric composition. For example, a composition having a higher percentage by weight of polymer is typically thicker and more viscous than a composition having a lower percentage by weight of polymer. A more viscous composition tends to flow more slowly. Therefore, a composition having a lower viscosity may be preferred in some instances.

In various embodiments, the molecular weight of the gel can be varied by any one of the many methods known in the art. The choice of method to vary molecular weight is typically determined by the composition of the gel (e.g., polymer versus non-polymer). For example in various embodiments, when the gel comprises one or more polymers, the degree of polymerization can be controlled by varying the amount of polymer initiators (e.g. benzoyl peroxide), organic solvents or activator (e.g. DMPT), crosslinking agents, polymerization agent, and/or reaction time.

Suitable gel polymers may be soluble in an organic solvent. The solubility of a polymer in a solvent varies depending on the crystallinity, hydrophobicity, hydrogen-bonding and molecular weight of the polymer. Lower molecular weight polymers will normally dissolve more readily in an organic solvent than high-molecular weight polymers. A polymeric gel, which includes a high molecular weight polymer, tends to coagulate or solidify more quickly than a polymeric composition, which includes a low-molecular weight polymer. Polymeric gel formulations, which include high molecular weight polymers, also tend to have a higher solution viscosity than a polymeric gel, which include a low-molecular weight polymer.

When the gel is designed to be a flowable gel, it can vary from low viscosity, similar to that of water, to a high viscosity, similar to that of a paste, depending on the molecular weight and concentration of the polymer used in the gel. The viscosity of the gel can be varied such that the polymeric composition can be applied to a patient's tissues by any convenient technique, for example, by brushing, spraying, dripping, injecting, or painting. Different viscosities of the gel will depend on the technique used to apply the composition.

In various embodiments, the gel has an inherent viscosity (abbreviated as “I.V.” and units are in deciliters/gram), which is a measure of the gel's molecular weight and degradation time (e.g., a gel with a high inherent viscosity has a higher molecular weight and longer degradation time). Typically, a gel with a high molecular weight provides a stronger matrix and the matrix takes more time to degrade. In contrast, a gel with a low molecular weight degrades more quickly and provides a softer matrix. In various embodiments, the gel has a molecular weight, as shown by the inherent viscosity, from about 0.10 dL/g to about 1.2 dL/g or from about 0.10 dL/g to about 0.40 dL/g.

In various embodiments, the gel can have a viscosity of about 300 to about 5,000 centipoise (cp). In other embodiments, the gel can have a viscosity of from about 5 to about 300 cps, from about 10 cps to about 50 cps, from about 15 cps to about 75 cps at room temperature. The gel may optionally have a viscosity enhancing agent such as, for example, hydroxypropyl cellulose, hydroxypropyl methylcellulose, hydroxyethyl methylcellulose, carboxymethylcellulose and salts thereof, Carbopol, poly-(hydroxyethylmethacrylate), poly-(methoxyethylmethacrylate), poly(methoxyethoxyethyl methacrylate), polymethylmethacrylate (PMMA), methylmethacrylate (MMA), gelatin, polyvinyl alcohols, propylene glycol, PEG 200, PEG 300, PEG 400, PEG 500, PEG 600, PEG 700, PEG 800, PEG 900, PEG 1000, PEG 1450, PEG 3350, PEG 4500, PEG 8000 or combinations thereof.

In various embodiments, when a polymer is employed in the gel, the polymeric composition includes about 10 wt % to about 90 wt % or about 30 wt % to about 60 wt % of the polymer.

In various embodiments, the gel is a hydrogel made of high molecular weight biocompatible elastomeric polymers of synthetic or natural origin. A desirable property for the hydrogel to have is the ability to respond rapidly to mechanical stresses, particularly shears and loads, in the human body.

Hydrogels obtained from natural sources are particularly appealing because they are more likely to be biodegradable and biocompatible for in vivo applications. Suitable hydrogels include natural hydrogels, such as, for example, gelatin, collagen, silk, elastin, fibrin and polysaccharide-derived polymers like agarose, and chitosan, glucomannan gel, hyaluronic acid, polysaccharides, such as cross-linked carboxyl-containing polysaccharides, or a combination thereof. Synthetic hydrogels include, but are not limited to those formed from polyvinyl alcohol, acrylamides such as polyacrylic acid and poly(acrylonitrile-acrylic acid), polyurethanes, polyethylene glycol (e.g., PEG 3350, PEG 4500, PEG 8000), silicone, polyolefins such as polyisobutylene and polyisoprene, copolymers of silicone and polyurethane, neoprene, nitrile, vulcanized rubber, poly(N-vinyl-2-pyrrolidone), acrylates such as poly(2-hydroxy ethyl methacrylate) and copolymers of acrylates with N-vinyl pyrolidone, N-vinyl lactams, polyacrylonitrile or combinations thereof. The hydrogel materials may further be cross-linked to provide further strength as needed. Examples of different types of polyurethanes include thermoplastic or thermoset polyurethanes, aliphatic or aromatic polyurethanes, polyetherurethane, polycarbonate-urethane or silicone polyether-urethane, or a combination thereof.

In various embodiments, rather than directly admixing the therapeutic agents into the gel, microspheres may be dispersed within the gel, the microspheres being loaded with at least one analgesic agent and/or at least one anti-inflammatory agent. In one embodiment, the microspheres provide for a sustained release of the at least one analgesic and/or anti-inflammatory agent. In yet another embodiment, the gel, which is biodegradable, prevents the microspheres from releasing the at least one analgesic and/or anti-inflammatory agent; the microspheres thus do not release the at least one analgesic and/or anti-inflammatory agent until it has been released from the gel. For example, a gel may be deployed around a target tissue site (e.g., a nerve root). Dispersed within the gel are a plurality of microspheres that encapsulate the desired therapeutic agent. Certain of these microspheres degrade once released from the gel, thus releasing the at least one analgesic and/or anti-inflammatory agent. The analgesic and/or anti-inflammatory agent may be placed into separate microspheres and then the microspheres combined, or the active ingredients can first be combined and then placed into the microspheres together.

Microspheres, much like a fluid, may disperse relatively quickly, depending upon the surrounding tissue type, and hence disperse the at least one analgesic agent and at least one anti-inflammatory agent. In some embodiments, the diameter of the microspheres range from about 10 microns in diameter to about 200 microns in diameter. In some embodiments they range from about 20 to 120 microns in diameters. Methods for making microspheres include but are not limited to solvent evaporation, phase separation and fluidized bed coating. In some situations, this may be desirable; in others, it may be more desirable to keep the at least one analgesic agent and at least one anti-inflammatory agent tightly constrained to a well-defined target site.

The present invention also contemplates the use of adherent gels to so constrain dispersal of the therapeutic agent. These gels may be deployed, for example, in a disc space, in a spinal canal, or in surrounding tissue.

### Cannulas and Needles

It will be appreciated by those with skill in the art that the depot can be administered to the target site using a “cannula” or “needle” that can be a part of a drug delivery device e.g., a syringe, a gun drug delivery device, or any medical device suitable for the application of a drug to a targeted organ or anatomic region. The cannula or needle of the drug depot device is designed to cause minimal physical and psychological trauma to the patient.

Cannulas or needles include tubes that may be made from materials, such as for example, polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. The cannula or needle may optionally include one or more tapered regions. In various embodiments, the cannula or needle may be beveled. The cannula or needle may also have a tip style vital for accurate treatment of the patient depending on the site for implantation. Examples of tip styles include, for example, Trephine, Cournand, Veress, Huber, Seldinger, Chiba, Francine, Bias, Crawford, deflected tips, Hustead, Lancet, or Tuohey. In various embodiments, the cannula or needle may also be non-coring and have a sheath covering it to avoid unwanted needle sticks.

The dimensions of the hollow cannula or needle, among other things, will depend on the site for implantation. For example, the width of the epidural space is only about 3-5 mm for the thoracic region and about 5-7 mm for the lumbar region. Thus, the needle or cannula, in various embodiments, can be designed for these specific areas. In various embodiments, the cannula or needle may be inserted using a transforaminal approach in the spinal foramen space, for example, along an inflamed nerve root and the drug depot implanted at this site for treating the condition. Typically, the transforaminal approach involves approaching the intervertebral space through the intervertebral foramina.

Some examples of lengths of the cannula or needle may include, but are not limited to, from about 50 to 150 mm in length, for example, about 65 mm for epidural pediatric use, about 85 mm for a standard adult and about 110 mm for an obese adult patient. The thickness of the cannula or needle will also depend on the site of implantation. In various embodiments, the thickness includes, but is not limited to, from about 0.05 to about 1.655. The gauge of the cannula or needle may be the widest or smallest diameter or a diameter in between for insertion into a human or animal body. The widest diameter is typically about 14 gauge, while the smallest diameter is about 25 gauge. In various embodiments the gauge of the needle or cannula is about 18 to about 22 gauge.

In various embodiments, like the drug depot and/or gel, the cannula or needle includes dose radiographic markers that indicate location at or near the site beneath the skin, so that the user may accurately position the depot at or near the site using any of the numerous diagnostic imaging procedures. Such diagnostic imaging procedures include, for example, X-ray imaging or fluoroscopy. Examples of such radiographic markers include, but are not limited to, barium, calcium phosphate, and/or metal beads or particles.

In various embodiments, the needle or cannula may include a transparent or translucent portion that can be visualizable by ultrasound, fluoroscopy, x-ray, or other imaging techniques. In such embodiments, the transparent or translucent portion may include a radiopaque material or ultrasound responsive topography that increases the contrast of the needle or cannula relative to the absence of the material or topography.

### Sterilization

The drug depot, and/or medical device to administer the drug may be sterilizable. In various embodiments, one or more components of the drug depot, and/or medical device to administer the drug are sterilized by radiation in a terminal sterilization step in the final packaging. Terminal sterilization of a product provides greater assurance of sterility than from processes such as an aseptic process, which require individual product components to be sterilized separately and the final package assembled in a sterile environment.

Typically, in various embodiments, gamma radiation is used in the terminal sterilization step, which involves utilizing ionizing energy from gamma rays that penetrates deeply in the device. Gamma rays are highly effective in killing microorganisms, they leave no residues nor have sufficient energy to impart radioactivity to the device. Gamma rays can be employed when the device is in the package and gamma sterilization does not require high pressures or vacuum conditions, thus, package seals and other components are not stressed. In addition, gamma radiation eliminates the need for permeable packaging materials.

In various embodiments, electron beam (e-beam) radiation may be used to sterilize one or more components of the device. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity. E-beam sterilization may be used, for example, when the drug depot is included in a gel.

Other methods may also be used to sterilize the depot and/or one or more components of the device, including, but not limited to, gas sterilization, such as, for example, with ethylene oxide or steam sterilization.

### Kits

In various embodiments, a kit is provided that may include additional parts along with the drug depot and/or medical device combined together to be used to implant the drug depot (e.g., pellet). The kit may include the drug depot device in a first compartment. The second compartment may include a canister holding the drug depot and any other instruments needed for the localized drug delivery. A third compartment may include gloves, drapes, wound dressings and other procedural supplies for maintaining sterility of the implanting process, as well as an instruction booklet. A fourth compartment may include additional cannulas and/or needles. A fifth compartment may include the agent for radiographic imaging. Each tool may be separately packaged in a plastic pouch that is radiation sterilized. A cover of the kit may include illustrations of the implanting procedure and a clear plastic cover may be placed over the compartments to maintain sterility.

### Administration

In various embodiments, the analgesic and/or anti-inflammatory agent may be parenterally administered. The term “parenteral” as used herein refers to modes of administration, which bypass the gastrointestinal tract, and includes for example, localized intravenous, intramuscular, continuous or intermittent infusion, intraperitoneal, intrasternal, subcutaneous, intra-operatively, intrathecally, intradiscally, peridiscally, epidurally, perispinally, intraarticular injection or combinations thereof.

The method of the present application comprises inserting a cannula at or near a target tissue site and implanting the drug depot at the target site beneath the skin of the patient and brushing, dripping, spraying, injecting, or painting the gel in the target site to hold or have the drug depot adhere to the target site. In this way unwanted migration of the drug depot away from the target site is reduced or eliminated.

In various embodiments, because the analgesic and/or anti-inflammatory agent is locally administered, therapeutically effective doses may be less than doses administered by other routes (oral, topical, etc.). For example, the drug dose delivered from the drug depot may be, for example, 10%, 20%, 30%, 40%, 50%, 60%, 70%, 80%, 90%, 95%, 99%, or 99.9% less than the oral dosage or injectable dose. In turn, systemic side effects, such as for example, liver transaminase elevations, hepatitis, liver failure, myopathy, constipation, etc. may be reduced or eliminated.

In various embodiments, to administer the gel having the drug depot dispersed therein to the desired site, first the cannula or needle can be inserted through the skin and soft tissue down to the target tissue site and the gel administered (e.g., brushed, dripped, injected, or painted, etc.) at or near the target site. In those embodiments where the drug depot is separate from the gel, first the cannula or needle can be inserted through the skin and soft tissue down to the site of injection and one or more base layer(s) of gel can be administered to the target site. Following administration of the one or more base layer(s), the drug depot can be implanted on or in the base layer(s) so that the gel can hold the depot in place or reduce migration. If required a subsequent layer or layers of gel can be applied on the drug depot to surround the depot and further hold it in place. Alternatively, the drug depot may be implanted first and then the gel placed (e.g., brushed, dripped, injected, or painted, etc.) around the drug depot to hold it in place. By using the gel, accurate and precise implantation of a drug depot can be accomplished with minimal physical and psychological trauma to the patient. The gel also avoids the need to suture the drug depot to the target site reducing physical and psychological trauma to the patient.

In various embodiments, when the target site comprises a spinal region, a portion of fluid (e.g., spinal fluid, etc.) can be withdrawn from the target site through the cannula or needle first and then the depot administered (e.g., placed, dripped, injected, or implanted, etc.). The target site will re-hydrate (e.g., replenishment of fluid) and this aqueous environment will cause the drug to be released from the depot.

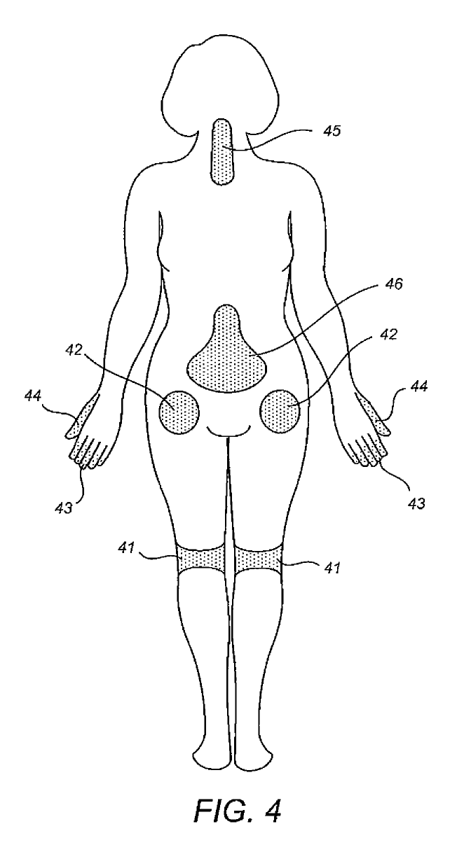

FIG. 4 illustrates a number of common locations within a patient that may be sites at which inflammation and/or pain may occur. It will be recognized that the locations illustrated in FIG. 4 are merely exemplary of the many different locations within a patient that may be the sites of inflammation and/or pain. For example, inflammation and/or pain may occur at a patient's knees 21, hips 22, fingers 23, thumbs 24, neck 25, and spine 26. These are also areas where the heat, cold or another suitable form of energy, e.g., ultrasound energy, can be applied thereto to cause bolus release of the analgesic and/or anti-inflammatory agent from the drug depot and provide the patient with the “extra dose.”

The analgesic and/or anti-inflammatory agent may be formed in a drug depot and administered with a suitable pharmaceutical carrier that may be solid or liquid, and placed in the appropriate form for parenteral or other administration as desired. As persons of ordinary skill are aware, known carriers include but are not limited to water, gelatin, lactose, starches, stearic acid, magnesium stearate, sicaryl alcohol, talc, vegetable oils, benzyl alcohols, gums, waxes, propylene glycol, polyalkylene glycols and other known carriers.

Another embodiment provides a method for treating a mammal suffering from pain and/or inflammation, said method comprising administering a therapeutically effective amount of at least one analgesic and/or anti-inflammatory agent at a target site beneath the skin at or near the target site. The at least analgesic and/or anti-inflammatory agent may for example be administered locally to the target tissue site as a drug depot.

In some embodiments, the therapeutically effective dosage amount (e.g., analgesic and/or anti-inflammatory agent dose) and the release rate profile are sufficient to reduce inflammation and/or pain following surgery, chronic inflammatory diseases, chronic inflammatory bowel disease, bursitis, osteoarthritis, osteolysis, tendonitis, sciatica, herniated discs, stenosis, myopathy, spondilothesis, lower back pain, facet pain, carpal tunnel syndrome, tarsal tunnel syndrome, failed back pain or the like for a period of at least one day, for example, 1-90 days, 1-10 days, 1-3 days, 3-7 days, 3-10 days, 3-12 days; 3-14 days, 7-10 days, 7-14 days, 7-21 days, 7-30 days, 7-50 days, 7-90 days, 7-140 days, 14-140 days, 3 days to 135 days, 3 days to 150 days, or 3 days to 6 months.

In some embodiments there is a composition useful for the treatment of inflammation comprising an effective amount of at least one analgesic and/or anti-inflammatory agent that is capable of being locally administered to a target tissue site. By way of example, they may be administered locally to the foraminal spine, the epidural space or the intrathecal space of a spinal cord. Exemplary administration routes include but are not limited to drug pumps, one or more local injections, polymer releases and combinations thereof.

In some embodiments, the at least one analgesic and/or anti-inflammatory agent is administered parenterally, e.g., by injection. In some embodiments, the injection is intrathecal, which refers to an injection into the spinal canal (intrathecal space surrounding the spinal cord). An injection may also be into a muscle or other tissue. In other embodiments, the analgesic and/or anti-inflammatory agent is administered by placement into an open patient cavity during surgery.

In some embodiments, the formulation is implantable into a surgical site at the time of surgery. The active ingredients may then be released from the depot via diffusion in a sustained fashion over a period of time, e.g., 1-10 days, 3-10 days, 3-15 days, 5-10 days or 7-10 days post surgery. The drug depot allows for bolus doses from the reversible phase transition polymer, as well.

In some embodiments, the drug depot may release 5%, 10%, 15%, 20%, 25%, 30%, 40%, 50%, 60%, 70%, 80%, 90%, 95%, or 99% of the at least one the analgesic and/or anti-inflammatory agent or pharmaceutically acceptable salt thereof relative to a total amount of at least one the analgesic and/or anti-inflammatory agent loaded in the drug depot over a period of 3 to 12 days, 5 to 10 days or 7 to 10 days after the drug depot is administered to the target tissue site. In some embodiments, the active ingredient may provide longer duration of pain and/or inflammation relief for chronic diseases/conditions as discussed above with release of one or more drugs up to 6 months or 1 year (e.g., 90, 100, 135, 150, 180 days or longer).

In various embodiments, an implantable drug depot useful for reducing, preventing or treating pain and/or inflammation is provided in a patient in need of such treatment, the implantable drug depot comprising a therapeutically effective amount of the analgesic and/or anti-inflammatory agent or pharmaceutically acceptable salts thereof, the depot being implantable at a site beneath the skin to reduce, prevent or treat pain and/or inflammation, following surgery, or resulting from chronic inflammatory diseases, chronic inflammatory bowel disease, bursitis, osteoarthritis, osteolysis, tendonitis, sciatica, herniated discs, stenosis, myopathy, spondilothesis, lower back pain, facet pain, carpal tunnel syndrome, tarsal tunnel syndrome, failed back pain or the like, wherein the drug depot (i) comprises one or more immediate release layer(s) that is capable of releasing about 5% to about 20% of the analgesic and/or anti-inflammatory agent or pharmaceutically acceptable salts thereof relative to a total amount of the analgesic and/or anti-inflammatory agent or pharmaceutically acceptable salt thereof loaded in the drug depot over a first period of up to 48 hours and (ii) one or more sustain release layer(s) that is capable of releasing about 21% to about 99% of the analgesic and/or anti-inflammatory agent or pharmaceutically acceptable salt thereof relative to a total amount of the analgesic and/or anti-inflammatory agent or pharmaceutically acceptable salt thereof loaded in the drug depot over a subsequent period of up to 3 days to 6 months.

By way of non-limiting example, the target tissue site may comprise at least one muscle, ligament, tendon, cartilage, spinal disc, spinal foraminal space near the spinal nerve root, facet or spinal canal. Also by way of example, the inflammation may be associated with orthopedic or spine surgery or a combination thereof. By way of further example, the surgery may be arthroscopic surgery, an excision of a mass, hernia repair, spinal fusion, thoracic, cervical, or lumbar surgery, pelvic surgery or a combination thereof. In some embodiments, the active ingredient may provide longer duration of pain and/or inflammation relief for chronic diseases/conditions as discussed above with release of one or more drugs over a period of 1-90 days, 1-10 days, 1-3 days, 3-7 days, 3-12 days; 3-14 days, 7-10 days, 7-14 days, 7-21 days, 7-30 days, 7-50 days, 7-90 days, 7-140 days, 14-140 days, 3 days to 135 days, 3 days to 150 days, or 3 days to 6 months.

In some embodiments, the at least one the analgesic and/or anti-inflammatory agent or pharmaceutically acceptable salt thereof is encapsulated in a plurality of depots comprising microparticles, microspheres, microcapsules, and/or microfibers suspended in a gel.

In some embodiments, a method is provided of inhibiting pain and/or inflammation following surgery, or resulting from chronic inflammatory diseases, chronic inflammatory bowel disease, bursitis, osteoarthritis, osteolysis, tendonitis, sciatica, herniated discs, stenosis, myopathy, spondilothesis, lower back pain, facet pain, carpal tunnel syndrome, tarsal tunnel syndrome, failed back pain or the like in a patient in need of such treatment, the method comprising delivering one or more biodegradable drug depots comprising a therapeutically effective amount of at least one analgesic and/or anti-inflammatory agent or pharmaceutically acceptable salt thereof to a target tissue site beneath the skin before, during or after surgery, wherein the drug depot releases an effective amount of at least one analgesic and/or anti-inflammatory agent or pharmaceutically acceptable salt thereof over a period of 3 days to 6 months.

In some embodiments, an implantable drug depot is provided, wherein the drug depot (i) comprises one or more immediate release layer(s) that comprise a reversible phase transition polymer that releases a bolus dose of at least one analgesic and/or anti-inflammatory agent or pharmaceutically acceptable salt thereof, when heat, cold or another suitable form of energy, e.g., ultrasound energy, is applied to it at a site beneath the skin and (ii) one or more sustain release layer(s) that releases an effective amount of at least one analgesic and/or anti-inflammatory agent or pharmaceutically acceptable salt thereof over a period of 3 to 12 days or 5 to 10 days or 7 to 10 days or 3 days to 6 months. By way of example, in the drug depot, the one or more sustained release layers comprising poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone, or a combination thereof and the reversible phase transition material in an immediate release layer comprises paraffin waxes, poloxamers, polylactones, poly(N-isopropylacrylamide) homopolymer, poly(N-isopropylacrylamide)acrylamide copolymer, copolymer of poly(N-isopropylacrylamide) containing silane monomers selected from [3-(methacryloyloxy)propyl]trimethoxysilane, [2-(methacryloyloxy)ethoxy]-trimethylsilane and methacryloyloxy)trimethylsilane, copolymer of poly(hydroxypropyl methacrylamide), dicarboxymethylaminopropyl methacrylamide, xyloglucan, ethyl(hydroxyethyl)cellulose, poly(ethyleneoxide-b-propylene oxide-b-ethylene oxide) and its copolymers, poly(ethylene oxide)/(D,L-lactic acid-co-glycolic acid)copolymers, combinations of chitosan and polyol salts, poly(silamine), and poly(organophosphazene) or a combination thereof.

### Method of Making

In various embodiments, the drug depot comprising the active ingredients (e.g., anti-inflammatory agent) can be made by combining a biocompatible polymer (reversible phase transition polymer(s) and/or sustained release polymer(s)) and a therapeutically effective amount of the active ingredients or pharmaceutically acceptable salts thereof and forming the implantable drug depot from the combination.

Various techniques are available for forming at least a portion of a drug depot from the biocompatible polymer(s), therapeutic agent(s), and optional materials, including solution processing techniques and/or thermoplastic processing techniques. Where solution processing techniques are used, a solvent system is typically selected that contains one or more solvent species. The solvent system is generally a good solvent for at least one component of interest, for example, biocompatible polymer and/or therapeutic agent. The particular solvent species that make up the solvent system can also be selected based on other characteristics, including drying rate and surface tension.

Solution processing techniques include solvent casting techniques, spin coating techniques, web coating techniques, solvent spraying techniques, dipping techniques, techniques involving coating via mechanical suspension, including air suspension (e.g., fluidized coating), ink jet techniques and electrostatic techniques. Where appropriate, techniques such as those listed above can be repeated or combined to build up the depot to obtain the desired release rate and desired thickness.

In various embodiments, a solution containing solvent and biocompatible polymer are combined and placed in a mold of the desired size and shape. In this way, polymeric regions, including barrier layers, lubricious layers, and so forth can be formed. If desired, the solution can further comprise, one or more of the following: other therapeutic agent(s) and other optional additives such as radiographic agent(s), etc. in dissolved or dispersed form. This results in a polymeric matrix region containing these species after solvent removal. In other embodiments, a solution containing solvent with dissolved or dispersed therapeutic agent is applied to a pre-existing polymeric region, which can be formed using a variety of techniques including solution processing and thermoplastic processing techniques, whereupon the therapeutic agent is imbibed into the polymeric region.

Thermoplastic processing techniques for forming the depot or portions thereof include molding techniques (for example, injection molding, rotational molding, and so forth), extrusion techniques (for example, extrusion, co-extrusion, multi-layer extrusion, and so forth) and casting.

Thermoplastic processing in accordance with various embodiments comprises mixing or compounding, in one or more stages, the biocompatible polymer(s) and one or more of the following: the active ingredients (e.g., alpha agonist), optional additional therapeutic agent(s), radiographic agent(s), and so forth. The resulting mixture is then shaped into an implantable drug depot. The mixing and shaping operations may be performed using any of the conventional devices known in the art for such purposes.

During thermoplastic processing, there exists the potential for the therapeutic agent(s) to degrade, for example, due to elevated temperatures and/or mechanical shear that are associated with such processing. For example, certain therapeutic agents may undergo substantial degradation under ordinary thermoplastic processing conditions. Hence, processing is preferably performed under modified conditions, which prevent the substantial degradation of the therapeutic agent(s). Although it is understood that some degradation may be unavoidable during thermoplastic processing, degradation is generally limited to 10% or less. Among the processing conditions that may be controlled during processing to avoid substantial degradation of the therapeutic agent(s) are temperature, applied shear rate, applied shear stress, residence time of the mixture containing the therapeutic agent, and the technique by which the polymeric material and the therapeutic agent(s) are mixed.

Mixing or compounding biocompatible polymer with therapeutic agent(s) and any additional additives to form a substantially homogenous mixture thereof may be performed with any device known in the art and conventionally used for mixing polymeric materials with additives.

Where thermoplastic materials are employed, a polymer melt may be formed by heating the biocompatible polymer, which can be mixed with various additives (e.g., therapeutic agent(s), inactive ingredients, etc.) to form a mixture. A common way of doing so is to apply mechanical shear to a mixture of the biocompatible polymer(s) and additive(s). Devices in which the biocompatible polymer(s) and additive(s) may be mixed in this fashion include devices such as single screw extruders, twin screw extruders, banbury mixers, high-speed mixers, ross kettles, and so forth.

Any of the biocompatible polymer(s) and various additives may be premixed prior to a final thermoplastic mixing and shaping process, if desired (e.g., to prevent substantial degradation of the therapeutic agent among other reasons).

For example, in various embodiments, a biocompatible polymer is precompounded with a radiographic agent (e.g., radio-opacifying agent) under conditions of temperature and mechanical shear that would result in substantial degradation of the therapeutic agent, if it were present. This precompounded material is then mixed with therapeutic agent (e.g., alpha agonist) under conditions of lower temperature and mechanical shear, and the resulting mixture is shaped into the active ingredient containing drug depot. Conversely, in another embodiment, the biocompatible polymer can be precompounded with the therapeutic agent under conditions of reduced temperature and mechanical shear. This precompounded material is then mixed with, for example, a radio-opacifying agent, also under conditions of reduced temperature and mechanical shear, and the resulting mixture is shaped into the drug depot.

The conditions used to achieve a mixture of the biocompatible polymer and therapeutic agent and other additives will depend on a number of factors including, for example, the specific biocompatible polymer(s) and additive(s) used, as well as the type of mixing device used.

As an example, different biocompatible polymers will typically soften to facilitate mixing at different temperatures. For instance, where a depot is formed comprising PLGA or PLA polymer, a radio-opacifying agent (e.g., bismuth subcarbonate), and a therapeutic agent prone to degradation by heat and/or mechanical shear (e.g., clonidine), in various embodiments, the PGLA or PLA can be premixed with the radio-opacifying agent at temperatures of about, for example, 150° C. to 170° C. The therapeutic agent is then combined with the premixed composition and subjected to further thermoplastic processing at conditions of temperature and mechanical shear that are substantially lower than is typical for PGLA or PLA compositions. For example, where extruders are used, barrel temperature, volumetric output are typically controlled to limit the shear and therefore to prevent substantial degradation of the therapeutic agent(s). For instance, the therapeutic agent and premixed composition can be mixed/compounded using a twin screw extruder at substantially lower temperatures (e.g., 100-105° C.), and using substantially reduced volumetric output (e.g., less than 30% of full capacity, which generally corresponds to a volumetric output of less than 200 cc/min). It is noted that this processing temperature is well below the melting points of certain active ingredients, such as an anti-inflammatory and/or analgesic (e.g., clonidine) because processing at or above these temperatures will result in substantial therapeutic agent degradation. It is further noted that in certain embodiments, the processing temperature will be below the melting point of all bioactive compounds within the composition, including the therapeutic agent. After compounding, the resulting depot is shaped into the desired form, also under conditions of reduced temperature and shear.

In other embodiments, biodegradable polymer(s) and one or more therapeutic agents are premixed using non-thermoplastic techniques. For example, the biocompatible polymer can be dissolved in a solvent system containing one or more solvent species. Any desired agents (for example, a radio-opacifying agent, a therapeutic agent, or both radio-opacifying agent and therapeutic agent) can also be dissolved or dispersed in the solvents system. Solvent is then removed from the resulting solution/dispersion, forming a solid material. The resulting solid material can then be granulated for further thermoplastic processing (for example, extrusion) if desired.

As another example, the therapeutic agent can be dissolved or dispersed in a solvent system, which is then applied to a pre-existing drug depot (the pre-existing drug depot can be formed using a variety of techniques including solution and thermoplastic processing techniques, and it can comprise a variety of additives including a radio-opacifying agent and/or viscosity enhancing agent), whereupon the therapeutic agent is imbibed on or in the drug depot. As above, the resulting solid material can then be granulated for further processing, if desired.

Typically, an extrusion processes may be used to form the drug depot comprising a biocompatible polymer(s), therapeutic agent(s) and radio-opacifying agent(s). Co-extrusion may also be employed, which is a shaping process that can be used to produce a drug depot comprising the same or different layers or regions (for example, a structure comprising one or more polymeric matrix layers or regions that have permeability to fluids to allow immediate and/or sustained drug release). Multi-region depots can also be formed by other processing and shaping techniques such as co-injection or sequential injection molding technology.

In various embodiments, the depot that may emerge from the thermoplastic processing (e.g., pellet, strip, etc.) is cooled. Examples of cooling processes include air cooling and/or immersion in a cooling bath. In some embodiments, a water bath is used to cool the extruded depot. However, where a water-soluble therapeutic agent such as an active ingredient is used, the immersion time should be held to a minimum to avoid unnecessary loss of therapeutic agent into the bath.

In various embodiments, immediate removal of water or moisture by use of ambient or warm air jets after exiting the bath will also prevent re-crystallization of the drug on the depot surface, thus controlling or minimizing a high drug dose “initial burst” or “bolus dose” upon implantation or insertion if this is release profile is not desired.

In various embodiments, the drug depot can be prepared by mixing or spraying the drug with the polymer and then molding the depot to the desired shape. In various embodiments, active ingredients are used and mixed or sprayed with the PLGA or PEG550 polymer, and the resulting depot may be formed by extrusion and dried.

The drug depot may also be made by combining a biocompatible polymer and a therapeutically effective amount of at least one analgesic and/or anti-inflammatory agent or pharmaceutically acceptable salt thereof and forming the implantable drug depot from the combination.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. An implantable drug depot useful for reducing, preventing or treating pain and/or inflammation in a patient in need of such treatment, the implantable drug depot being implantable at a site beneath the skin and comprising an effective amount of an analgesic muscle relaxant, and/or an anti-inflammatory agent disposed within a reversible phase transition material of the drug depot, wherein the reversible phase transition material is capable of releasing a bolus dose of the analgesic, muscle relaxant and/or the anti-inflammatory agent when heat, cold or other suitable energy form is applied to the skin of a patient to reduce, prevent or treat pain and/or inflammation.

2. An implantable drug depot according to claim 1, wherein the drug depot releases an effective amount of the analgesic, muscle relaxant and/or the anti-inflammatory agent to treat post operative pain over a period of 3 to 10 days.

3. An implantable drug depot according to claim 1, wherein the site beneath the skin comprises at least one dermis, connective tissue, adipose tissue, muscle, ligament, tendon, cartilage, spinal disc, spinal foraminal space near the spinal nerve root, facet or synovial joint, or spinal canal.

4. An implantable drug depot according to claim 2, wherein the pain or inflammation is associated with hernia repair, orthopedic or spine surgery or a combination thereof.

5. An implantable drug depot according to claim 2, wherein the post operative pain is from one or more surgical procedures comprising arthroscopic surgery, an excision of a mass, spinal fusion, thoracic, cervical, or lumbar surgery, pelvic surgery or a combination thereof.

6. An implantable drug depot according to claim 1, wherein the drug depot comprises at least one biodegradable polymer comprising one or more of poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone, or a combination thereof and the reversible phase transition material comprises paraffin waxes, poloxamers, polylactones, poly(N-isopropylacrylamide) homopolymer, poly(N-isopropylacrylamide)acrylamide copolymer, copolymer of poly(N-isopropylacrylamide) containing silane monomers selected from [3-(methacryloyloxy)propyl]trimethoxysilane, [2-(methacryloyloxy)ethoxy]-trimethylsilane and methacryloyloxy)trimethylsilane, copolymer of poly(hydroxypropyl methacrylamide), dicarboxymethylaminopropyl methacrylamide, xyloglucan, ethyl(hydroxyethyl)cellulose, poly(ethyleneoxide-b-propylene oxide-b-ethylene oxide) and its copolymers, poly(ethylene oxide)/(D,L-lactic acid-co-glycolic acid)copolymers, combinations of chitosan and polyol salts, poly(silamine), and poly(organophosphazene) or a combination thereof.

7. An implantable drug depot according to claim 1, wherein the reversible phase transition polymer comprises at least one biodegradable polymer comprising paraffin waxes, poloxamers, polylactones, paraffin waxes, poly(N-isopropylacrylamide) homopolymer, poly(N-isopropylacrylamide)acrylamide copolymer, copolymer of poly(N-isopropylacrylamide) containing silane monomers selected from [3-(methacryloyloxy)propyl]trimethoxysilane, [2-(methacryloyloxy)ethoxy]-trimethylsilane and methacryloyloxy)trimethylsilane, copolymer of poly(hydroxypropyl methacrylamide), dicarboxymethylaminopropyl methacrylamide, xyloglucan, ethyl(hydroxyethyl)cellulose, poly(ethyleneoxide-b-propylene oxide-b-ethylene oxide) and its copolymers, poly(ethylene oxide)/(D,L-lactic acid-co-glycolic acid)copolymers, combinations of chitosan and polyol salts, poly(silamine), and poly(organophosphazene) or a combination thereof.

8. An implantable drug depot according to claim 1, wherein the reversible phase transition polymer is layered in the drug depot and the reversible phase transition polymer changes from solid to liquid to release the bolus dose of the analgesic, muscle relaxant and/or the anti-inflammatory agent when heat, cold or other energy form is applied to a site on the skin proximate to a site where the drug depot was implanted.

9. An implantable drug depot according to claim 1, wherein the reversible phase transition polymer comprises about 60% to 99% of the total weight % of the drug depot.

10. An implantable drug depot according to claim 1, wherein the heat applied to the skin is above about 40° C. to 45° C. to cause the reversible phase transition material in an implanted drug depot to change from solid to liquid or solid to semi-solid or semi-solid to liquid.

11. An implantable drug depot according to claim 1, wherein the cold applied to the skin is below 20° C. to 25° C. to cause the reversible phase transition material in an implanted drug depot to change from solid to liquid or solid to semi-solid or semi-solid to liquid.

12. An implantable drug depot according to claim 1, wherein the drug depot releases (i) a bolus dose of the analgesic and/or anti-inflammatory agent at a site beneath the skin over a period of up to 3 days and (ii) an effective amount of the analgesic and/or anti-inflammatory agent over a period of up to 6 months.

13. A drug depot useful for reducing, preventing or treating pain and/or inflammation in a patient in need of such treatment, the drug depot being implantable at a site beneath the skin of the patient and comprising an effective amount of an analgesic, muscle relaxant, and/or an anti-inflammatory agent disposed within a reversible phase transition polymer and a biodegradable polymer of the drug depot, wherein the reversible phase transition material is capable of releasing a bolus dose of the analgesic, muscle relaxant and/or the anti-inflammatory agent when heat, cold or other energy form is applied at or near the drug depot and the biodegradable polymer is capable of releasing the analgesic, muscle relaxant and/or the anti-inflammatory agent over at least one day to reduce, prevent or treat pain and/or inflammation.

14. A drug depot according to claim 13, wherein the drug depot is implanted within 1 mm of an epidermis, dermis, or subcutaneous tissue and heat, cold or other energy form is applied to the skin to cause release of the bolus dose of the analgesic, muscle relaxant and/or the anti-inflammatory agent.

15. A drug depot according to claim 13, wherein the drug depot is in the form of a pellet.

16. A drug depot according to claim 13, wherein the heat is applied for about 5 minutes to about 60 minutes to the skin of the patient near the drug depot when it is implanted at the site beneath the skin.

17. A drug depot according to claim 13, wherein the cold is applied for about 5 minutes to about 60 minutes to the skin of the patient near the drug depot when it is implanted at the site beneath the skin.

18. A method of treating or preventing pain and/or inflammation in a patient in need of such treatment, the method comprising implanting at a target tissue site beneath the skin of patient a biodegradable drug depot comprising an effective amount of an analgesic, muscle relaxant, and/or an anti-inflammatory agent disposed within a reversible phase transition material of the drug depot, wherein the reversible phase transition material is capable of releasing a bolus dose of the analgesic, muscle relaxant and/or the anti-inflammatory agent when heat, cold or other energy form is applied to or near the drug depot; and applying heat, cold or other energy form to or near the target tissue site where the drug depot is implanted to release the bolus dose of the analgesic, muscle relaxant and/or the anti-inflammatory agent to prevent or treat pain and/or inflammation.

19. A method according to claim 18, wherein the drug depot further comprises a biodegradable polymer that is capable of releasing the analgesic, muscle relaxant and/or the anti-inflammatory agent over at least one day to prevent or treat pain and/or inflammation.

20. A method according to claim 18, wherein (i) heat is applied to the skin of the patient and the heat is above about 40° C. to 45° C. to cause the reversible phase transition material to change from solid to liquid or solid to semi-solid or semi-solid to liquid to release the bolus dose or (ii) cold is applied to the skin of the patient and the cold is below about 20° C. to 25° C. to cause the reversible phase transition material to change from solid to liquid or solid to semi-solid or semi-solid to liquid to release the bolus dose.
